(19) United States
(12) Patent Application Publication
(10) Pub. No.: US 2021/0052598 A1
(43) Pub. Date: Feb. 25, 2021

(54) COMPOSITIONS AND METHODS FOR TREATING CANCER

(71) Applicant: Beth Israel Deaconess Medical Center, Inc., Boston, MA (US)

(72) Inventor: Wenyi Wei, Boston, MA (US)

(73) Assignee: Beth Israel Deaconess Medical Center, Inc., Boston, MA (US)

(21) Appl. No.: 16/977,375

(22) PCT Filed: Mar. 6, 2019
(86) PCT No.: PCT/US19/20924

§ 371 Date: Sep. 1, 2020

Related U.S. Application Data
(60) Provisional application No. 62/639,561, filed on Mar. 7, 2018.

(51) Int. Cl. A61K 31/551; A61K 31/4353; G01N 33/574

(52) U.S. Cl. CPC A61K 31/551; G01N 33/574; A61K 31/4353

(57) ABSTRACT

The present disclosure features compositions and methods of treating a cancer in a subject by administering to the subject a Skp2 inhibitor and an inhibitor of glycolytic metabolism (e.g., PKM2 inhibitor).

### CROSS-REFERENCE TO RELATED APPLICATION

This application claims the benefit of the following U.S. Provisional Application No. 62/639,561, filed Mar. 7, 2018, the entire contents of which are incorporated herein by reference.

### STATEMENT OF RIGHTS TO INVENTIONS MADE UNDER FEDERALLY SPONSORED RESEARCH

This invention was made with government support under Grant Nos. GM094777 and CA200573 awarded by the National Institutes of Health. The government has certain rights in the invention.

### BACKGROUND OF THE INVENTION

Whether glucose is predominantly metabolized via oxidative-phosphorylation or glycolysis differs between quiescent versus proliferating cells, including tumor cells. Given the high demand for biomacromolecules, including lipid, nucleotides and amino acids, to prepare for DNA replication and subsequent cell division, high rates of glycolysis and low rates of TCA cycle enable more flux of intermediates into the biomass synthesis pathways. Indeed, several lines of evidence advocate a bi-directional interplay between the cell cycle and metabolic machineries. On one hand, key metabolic enzymes are directly regulated in a cell cycle-dependent manner, such as PFKFB3 (6-phosphofructo-2-kinase/fructose-2, 6-bisphosphatase-3) by SCF^{GRR1}, SCF^{β-TRIP} and APC^{Cdh1}, HK2 (hexokinase 2) by cyclin D1, PFKP and PKM2 by CDK6/cyclin D3, and GLS1 by APC^{Cdh1}. On the other hand, disturbing metabolism also could compromise cell cycle progress. Improved methods for disrupting pathways critical for tumor metabolism.

### SUMMARY OF THE INVENTION

As described below, the present invention generally features compositions and methods of treating a cancer in a subject by administering to the subject a Skp2 inhibitor and an inhibitor of glycolytic metabolism (e.g., PKM2 inhibitor).

In one aspect, the invention provides a method of reducing neoplastic cell proliferation or survival involving contacting the cell with a Skp2 inhibitor and a Pyruvate kinase M2 (PKM2) inhibitor, thereby reducing neoplastic cell proliferation or survival.

In another aspect, the invention provides a method of reducing tumor growth involving contacting the tumor with a Skp2 inhibitor and a Pyruvate kinase M2 (PKM2) inhibitor, thereby reducing tumor growth.

In another aspect, the invention provides a method of treating cancer in a subject involving administering to the subject a Skp2 inhibitor and a Pyruvate kinase M2 (PKM2) inhibitor thereby treating cancer in the subject.

In another aspect, the invention provides a therapeutic combination for cancer therapy comprising a Skp2 inhibitor and a PKM2 inhibitor.

In another aspect, the invention provides a method of treating a selected subject having cancer involving administering a Skp2 inhibitor and an inhibitor of a glycolysis pathway enzyme to a selected subject, wherein the subject is selected by detecting an increased level of Skp2 and a decreased level of IDH1 and/or IDH2 in a biological sample of the subject, thereby treating the subject.

In various embodiments of any aspect delineated herein, the neoplastic cell or tumor displays one or more of increased glycolytic metabolism; reduced Tricarboxylic Acid (TCA) metabolism; increased lactate production; and/or reduced oxidative phosphorylation. In various embodiments of any aspect delineated herein, the neoplastic cell or tumor is characterized as Skp2^{high} and IDH1^{low}. In various embodiments, the neoplastic cell is a breast cancer, glioblastoma, or prostate cancer cell. In various embodiments, the tumor is breast cancer, glioblastoma, or prostate cancer.

In various embodiments of any aspect delineated herein, the subject has breast cancer, glioblastoma, or prostate cancer. In various embodiments, the subject's cancer displays one or more of increased glycolytic metabolism; reduced Tricarboxylic Acid (TCA) metabolism; increased lactate production; and/or reduced oxidative phosphorylation.

In various embodiments of any aspect delineated herein, the method involves detecting Skp2, p27, p21, Cyclin A, Cyclin E, IDH1, and/or IDH2 expression by immunoassay.

In various embodiments of any aspect delineated herein, the Skp2 inhibitor is one or more of a SKPin C1 and an inhibitory nucleic acid that targets Skp2 mRNA (e.g., for degradation). In various embodiments of any aspect delineated herein, the PKM2 inhibitor is one or more of 2 inhibitor compound 3k, DASA-58, and an inhibitory nucleic acid that targets PKM2 mRNA (e.g., for degradation). In various embodiments of any aspect delineated herein, the Skp2 inhibitor and PKM2 inhibitor are formulated together or separately Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “S-phase kinase-associated protein 2 (Skp2) polypeptide” is meant a polypeptide or fragment thereof having at least about 85%, or greater, amino acid identity to NCBI Accession No. NP_005974 (below) and having binding activity to cyclin A/E-CDK2, ubiquitin substrate recognizing activity, and/or oncogenic activity.

| | |
|---|---|
| 1 | mhrkhlqeip dlssnvatsf twgwdsskts ellsgmgvsa |
| | lekeepdsen ipqellsnlg |
| 61 | hpespprkrl kskgsdkdfv ivrrpklnre nfpgvswdsl |
| | pdelllgifs clclpellkv |
| 121 | sgvckrwyrl asdeslwqtl dltgknlhpd vtgrllsqgv |
| | iafrcprsfm dqplaehfsp |
| 181 | frvqhmdlsn svievstlhg ilsqcsklqn lsleglrlsd |
| | pivntlakns nlvrlnlsgc |
| 241 | sgfsefalqt llsscsrlde lnlswcfdft ekhvqvavah |
| | vsetitqlnl sgyrknlqks |
| 301 | dlstlvrrcp nlvhldlsds vmlkndcfge ffqlnylghl |
| | slsrcydiip etllelgeip |
| 361 | tlktlqvfgi vpdgt1q11k ealphlqinc shfttiarpt |
| | ignkknqeiw gikcrltlqk |
| 421 | pscl |

By “Skp2 nucleic acid molecule” is meant a polynucleotide encoding a Skp2 polypeptide. An exemplary Skp2 nucleic acid molecule sequence is provided at NCBI Accession No. NM_005983 (below):

| | |
|---|---|
| 1 | aattcccagc aggccttggg cctcagtgcg gccgcgaagc |
| | agagcgggct gtagagcctt |
| 61 | gcgcgcgcag tggggatgga acgttgctag gcttagcggg |
| | tctggctgct gggggcccga |
| 121 | gcagcacgct cggagccgcc gcgcgccaaa gcgggaatct |
| | gggaggcgag cagctctgca |
| 181 | gttaatgcac gtattttaaa ctcccgggcc tgcggacgct |
| | atgcacagga agcacctcca |
| 241 | ggagattcca gacctgagta gcaacgttgc caccagcttc |
| | acgtggggat gggattccag |
| 301 | caagacttct gaactgctgt caggcatggg ggtctccgcc |
| | ctggagaaag aggagcccga |
| 361 | cagtgagaac atcccccagg aactgctctc aaacctgggc |
| | cacccggaga gccccccacg |
| 421 | gaaacggctg aagagcaaag ggagtgacaa agactttgtg |
| | attgtccgca ggcctaagct |
| 481 | aaatcgagag aactttccag gtgtttcatg ggactccctt |
| | ccggatgagc tgctcttggg |
| 541 | aatcttttcc tgtctgtgcc tccctgagct gctaaaggtc |
| | tctggtgttt gtaagaggtg |
| 601 | gtatcgccta gcgtctgatg agtctctatg gcagacctta |
| | gacctcacag gtaaaaatct |
| 661 | gcacccggat gtgactggtc ggttgctgtc tcaaggggtg |
| | attgccttcc gctgcccacg |
| 721 | atcatttatg gaccaaccat tggctgaaca tttcagccct |
| | tttcgtgtac agcacatgga |
| 781 | cctatcgaac tcagttatag aagtgtccac cctccacggc |
| | atactgtctc agtgttccaa |
| 841 | gttgcagaat ctaagcctgg aaggcctgcg gctttcggat |
| | cccattgtca atactctcgc |
| 901 | aaaaaactca aatttagtgc gacttaacct ttctgggtgt |
| | tctggattct ctgaatttgc |
| 961 | cctgcagact ttgctaagca gctgttccag actggatgag |
| | ctgaacctct cctggtgttt |
| 1021 | tgatttcact gaaaagcatg tacaggtggc tgttgcgcat |
| | gtgtcagaga ccatcaccca |
| 1081 | gctgaatctt agcggctaca gaaagaatct ccagaaatca |
| | gatctctcta ctttagttag |
| 1141 | aagatgcccc aatcttgtcc atctagactt aagtgatagt |
| | gtcatgctaa agaatgactg |
| 1201 | ctttcaggaa tttttccagc tcaactacct ccaacaccta |
| | tcactcagtc ggtgctatga |
| 1261 | tataatacct gaaactttac ttgaacttgg agaaattccc |
| | acactaaaaa cactacaagt |
| 1321 | ttttggaatc gtgccagatg gtacccttca actgttaaag |
| | gaagcccttc ctcatctaca |
| 1381 | gattaattgc tcccatttca ccaccattgc caggccaact |
| | attggcaaca aaaagaacca |
| 1441 | ggagatatgg ggcatcaaat gccgactgac actgcaaaag |
| | cccagttgtc tatgaagtat |
| 1501 | ttattgcagg atggtgtctc ttctttagaa cagggaaaat |
| | aggcaggaag cccaattgct |
| 1561 | ggagtactta gctagtttta ttcttggttt tccctttgcc |
| | ttcattctgc aagtatacta |
| 1621 | gggagccatt tgagagggaa aactatgaaa tcttgctttt |
| | tgaaatgatt ctaaaagctt |
| 1681 | ctatcactgc tttgctctta agagccaaag ttgtaggcct |
| | tttgaaattt taggagagtg |
| 1741 | agcctataat ttcaagatac cttaaagagc aaaatttgag |
| | ccacctcttc caagtgccct |
| 1801 | tcttactaag tctattcaga atcaagctta aaaattacca |
| | ccagcaaaca atcttcatag |
| 1861 | cccatataac ttttatctat ttaattttat agtattgctt |
| | tataagacag cttagaagaa |
| 1921 | caataagcta tttgtattat gagctgaaca aaaagagaat |
| | cataggatag tagcgtctga |
| 1981 | ggccatcttt tctaggaata ggaaagagaa aaatgtattt |
| | gaattttgcc tttagatttg |
| 2041 | aaattaggtt aatagaaata agtaacccca tgtaattcac |
| | cttaaaactt aacaaaagac |
| 2101 | caaacattac aaaacccaga gatatagaat caatatagga |
| | tttgaaggcc cagcagacag |
| 2161 | ttttctatga caggttaatc tgaagtatcc tgtaatgttc |
| | attaagttac tgtgtttcca |
| 2221 | gaatctaaat tagatgagaa atataattgt ggttttctaa |
| | cttgataatc aaattatgtt |
| 2281 | aacatgggtc ctttagcttt taaaatgact tgctttgttt |
| | tagaaaggtg gtattaatcc |
| 2341 | actctctatt cttgaaaatt tggatgggag aattctgaag |
| | ttgcctgctg ttttccttta |
| 2401 | gcgctgaggt tcttaaggtt acttttatat tactctggaa |
| | tcaagtattt taaattgtat |
| 2461 | ttttttttta aatgatctct cagcaataat tgtttgaaac |
| | tatccatata taaggttatc |
| 2521 | agacctacag ttccctaaga ggaactgcat gttctcttca |
| | atcagaaata tacagtagaa |
| 2581 | gcaggtatat cttccatgca gtttcagtag taagcactac |
| | ttatacctac ataagagtta |
| 2641 | aaatccagat gtgggacctt ttgataccat cagtgatata |
| | tattttttta aactggtaca |
| 2701 | gagaagtgaa aagattaaat tctacttcta tttttttttt |
| | ttttttttga gacggagtct |
| 2761 | cgctctgtca ccaaggccgg agtgcagtgg tgcgatctcg |
| | gctcactgca agctccgcct |
| 2821 | cccaggttca cgtcattctc ctgcctcagc ctcccgacta |
| | gctgggacta caggcgccca |
| 2881 | ccaccacgcc cggctaattt ttttgtattt ttagtagaga |
| | cggggtttca ccatgttagc |
| 2941 | caggatggtc tcaatctcct gacctcatga tccgcccgtc |
| | ttggcctccc aaagtgctgg |
| 3001 | gattacaggc atgagcaact gcgcccagcc aaattctact |
| | tcttaaaaat cacaaaaact |
| 3061 | agtttaaatt gatgacttgt tcgtatgttc aaaatgtaac |
| | aacaaaaaaa gctaacacca |
| 3121 | gtcatttata ttaacttttt ttttttaaat caaaaattgt |
| | taatgttaga aacatactat |
| 3181 | gaagtgcctt tatctgctta gacctaagga agattttaaa |
| | gttgggttgc acaggaaatg |
| 3241 | atgatgcttc aatttcttaa tagttaaaaa gtgctaaata |
| | ctacttgaaa ttattgttta |
| 3301 | cagattagtg acaagagctg gggttaggat ccggttggac |
| | tctgacatcg gatgccctca |
| 3361 | aacatacaga acttccaaac tcaagtccag ccataagcta |
| | ttttgccaac atgtcagagt |
| 3421 | aatctgtatt tttgtatgtg atttctactt ttatagactt |
| | gttttaaaac aataaaacac |
| 3481 | atttttataa aaatgagtgc ttaaaaaaaa aaaaaaaaaa |

By “Skp2 Inhibitor” is meant an agent that inhibits Skp2 expression, function or activity. Exemplary Skp2 inhibitors are known in the art and described, for example, by Wu et al., Chem Biol. 2012 Dec. 21; 19(12): 1515-1524. Skp2 inhibitors include, but are not limited to, SKPin C1 (Tocris; also CAS 432001-69-9, Millipore Sigma) and Skp2 inhibitory nucleic acids.

By “Isocitrate dehydrogenase 1 (Idh1) polypeptide” is meant a polypeptide or fragment thereof having at least about 85%, or greater, amino acid identity to NCBI Accession No. NP_001269315 (below) and having isocitrate dehydrogenase activity (oxidative decarboxylation of isocitrate to α-ketoglutarate); nicotinamide adenine dinucleotide phosphate (NADP+) reducing activity (catalyzing NADP+ to NADPH); and/or the ability to homodimerize.

| | |
|---|---|
| 1 | mskkisggsv vemqgdemtr iiwelikekl ifpyveldlh |
| | sydlgienrd atndqvtkda |
| 61 | aeaikkhnvg vkcatitpde krveefklkq mwkspngtir |
| | nilggtvfre aiickniprl |
| 121 | vsgwvkpiii grhaygdqyr atdfvvpgpg kveitytpsd |
| | gtqkvtylvh nfeegggvam |
| 181 | gmynqdksie dfahssfqma lskgwplyls tkntilkkyd |
| | grfkdifqei ydkqyksgfe |
| 241 | aqkiwyehrl iddmvaqamk seggfiwack nydgdvqsds |
| | vaggygslgm mtsvlvcpdg |
| 301 | ktveaeaahg tvtrhyrmyq kgqetstnpi asifawtrgl |
| | ahrakldnnk elaffanale |
| 361 | evsietieag fmtkdlaaci kglpnvqrsd ylntfefmdk |
| | lgenlkikla qakl |

By “Idh1 nucleic acid molecule” is meant a polynucleotide encoding an Idh1 polypeptide. An exemplary Idh1 nucleic acid molecule sequence is provided at NCBI Accession No. NM_005896 (below):

| | |
|---|---|
| 1 | gggctgagga ggcggggcct gggaggggac aaagccggga |
| | agaggaaaag ctcggaccta |
| 61 | ccctgtggtc ccgggtttct gcagagtcta cttcagaagc |
| | ggaggcactg ggagtccggt |
| 121 | ttgggattgc caggctgtgg ttgtgagtct gagcttgtga |
| | gcggctgtgg cgccccaact |
| 181 | cttcgccagc atatcatccc ggcaggcgat aaactacatt |
| | cagttgagtc tgcaagactg |
| 241 | ggaggaactg gggtgataag aaatctattc actgtcaagg |
| | tttattgaag tcaaaatgtc |
| 301 | caaaaaaatc agtggcggtt ctgtggtaga gatgcaagga |
| | gatgaaatga cacgaatcat |
| 361 | ttgggaattg attaaagaga aactcatttt tccctacgtg |
| | gaattggatc tacatagcta |
| 421 | tgatttaggc atagagaatc gtgatgccac caacgaccaa |
| | gtcaccaagg atgctgcaga |
| 481 | agctataaag aagcataatg ttggcgtcaa atgtgccact |
| | atcactcctg atgagaagag |
| 541 | ggttgaggag ttcaagttga aacaaatgtg gaaatcacca |
| | aatggcacca tacgaaatat |
| 601 | tctgggtggc acggtcttca gagaagccat tatctgcaaa |
| | aatatccccc ggcttgtgag |
| 661 | tggatgggta aaacctatca tcataggtcg tcatgcttat |
| | ggggatcaat acagagcaac |
| 721 | tgattttgtt gttcctgggc ctggaaaagt agagataacc |
| | tacacaccaa gtgacggaac |
| 781 | ccaaaaggtg acatacctgg tacataactt tgaagaaggt |
| | ggtggtgttg ccatggggat |
| 841 | gtataatcaa gataagtcaa ttgaagattt tgcacacagt |
| | tccttccaaa tggctctgtc |
| 901 | taagggttgg cctttgtatc tgagcaccaa aaacactatt |
| | ctgaagaaat atgatgggcg |
| 961 | ttttaaagac atctttcagg agatatatga caagcagtac |
| | aagtcccagt ttgaagctca |
| 1021 | aaagatctgg tatgagcata ggctcatcga cgacatggtg |
| | gcccaagcta tgaaatcaga |
| 1081 | gggaggcttc atctgggcct gtaaaaacta tgatggtgac |
| | gtgcagtcgg actctgtggc |
| 1141 | ccaagggtat ggctctctcg gcatgatgac cagcgtgctg |
| | gtttgtccag atggcaagac |
| 1201 | agtagaagca gaggctgccc acgggactgt aacccgtcac |
| | taccgcatgt accagaaagg |
| 1261 | acaggagacg tccaccaatc ccattgcttc catttttgcc |
| | tggaccagag ggttagccca |
| 1321 | cagagcaaag cttgataaca ataaagagct tgccttcttt |
| | gcaaatgctt tggaagaagt |
| 1381 | ctctattgag acaattgagg ctggcttcat gaccaaggac |
| | ttggctgctt gcattaaagg |
| 1441 | tttacccaat gtgcaacgtt ctgactactt gaatacattt |
| | gagttcatgg ataaacttgg |
| 1501 | agaaaacttg aagatcaaac tagctcaggc caaactttaa |
| | gttcatacct gagctaagaa |
| 1561 | ggataattgt cttttggtaa ctaggtctac aggtttacat |
| | ttttctgtgt tacactcaag |
| 1621 | gataaaggca aaatcaattt tgtaatttgt ttagaagcca |
| | gagtttatct tttctataag |
| 1681 | tttacagcct ttttcttata tatacagtta ttgccacctt |
| | tgtgaacatg gcaagggact |
| 1741 | tttttacaat ttttatttta ttttctagta ccagcctagg |
| | aattcggtta gtactcattt |
| 1801 | gtattcactg tcactttttc tcatgttcta attataaatg |
| | accaaaatca agattgctca |
| 1861 | aaagggtaaa tgatagccac agtattgctc cctaaaatat |
| | gcataaagta gaaattcact |
| 1921 | gccttcccct cctgtccatg accttgggca cagggaagtt |
| | ctggtgtcat agatatcccg |
| 1981 | ttttgtgagg tagagctgtg cattaaactt gcacatgact |
| | ggaacgaagt atgagtgcaa |
| 2041 | ctcaaatgtg ttgaagatac tgcagtcatt tttgtaaaga |
| | ccttgctgaa tgtttccaat |
| 2101 | agactaaata ctgtttaggc cgcaggagag tttggaatcc |
| | ggaataaata ctacctggag |
| 2161 | gtttgtcctc tccatttttc tctttctcct cctggcctgg |
| | cctgaatatt atactactct |
| 2221 | aaatagcata tttcatccaa gtgcaataat gtaagctgaa |
| | tcttttttgg acttctgctg |
| 2281 | gcctgtttta tttcttttat ataaatgtga tttctcagaa |
| | attgatatta aacactatct |
| 2341 | tatcttctcc tgaactgttg attttaatta aaattaagtg |
| | ctaattacca ttaaaaaaaa |
| 2401 | aa |

By “Isocitrate dehydrogenase 2 (Idh2) polypeptide” is meant a polypeptide or fragment thereof having at least about 85%, or greater, amino acid identity to NCBI Accession No. NP_002159 (below) and having isocitrate dehydrogenase activity (oxidative decarboxylation of isocitrate to α-ketoglutarate); nicotinamide adenine dinucleotide phosphate (NADP+) reducing activity (catalyzing NADP+ to NADPH); and/or the ability to homodimerize.

| | |
|---|---|
| 1 | magylrvvrs lcrasgsrpa wapaaltapt sqeqprrhya |
| | dkrikvakpv vemdgdemtr |
| 61 | iiwgfikekl ilphvdiqlk yfdlglpnrd qtddqvtids |
| | alatqkysva vkcatitpde |
| 121 | arveefklkk mwkspngtir nilggtvfre piickniprl |
| | vpgwtkpiti grhahgdgyk |
| 181 | atdfvadrag tfkmvftpkd gsgvkewevy nfpaggvgmg |
| | myntdesisg fahscfqyai |
| 241 | qkkwplymst kntilkaydg rfkdifqeif dkhyktdfdk |
| | nkiwyehrli ddmvaqvlks |
| 301 | sggfvwackn ydgdvqsdil aqgfgslglm tsvlvcpdgk |
| | tieaeaahgt vtrhyrehqk |
| 361 | grptstnpia sifawtrgle hrgkldgnqd lirfaqmlek |
| | vcvetvesga mtkdlagcih |
| 421 | glsnvklneh flnttdfldt iksnldralg rq |

By “Idh2 nucleic acid molecule” is meant a polynucleotide encoding an Idh2 polypeptide. An exemplary Idh2 nucleic acid molecule sequence is provided at NCBI Accession No. NM_002168 (below):

| | |
|---|---|
| 1 | tccccggcaa ggcccaatgg ggcggcaggc ccggcagccc |
| | cgccccggtg gtgcccgcgc |
| 61 | ggccagcgcc cgccaggccc agcgttagcc cgcggccagg |
| | cagccgggag gagcggcgcg |
| 121 | cgctcggacc tctcccgccc tgctcgttcg ctctccagct |
| | tgggatggcc ggctacctgc |
| 181 | gggtcgtgcg ctcgctctgc agagcctcag gctcgcggcc |
| | ggcctgggcg ccggcggccc |
| 241 | tgacagcccc cacctcgcaa gagcagccgc ggcgccacta |
| | tgccgacaaa aggatcaagg |
| 301 | tggcgaagcc cgtggtggag atggatggtg atgagatgac |
| | ccgtattatc tggcagttca |
| 361 | tcaaggagaa gctcatcctg ccccacgtgg acatccagct |
| | aaagtatttt gacctcgggc |
| 421 | tcccaaaccg tgaccagact gatgaccagg tcaccattga |
| | ctctgcactg gccacccaga |
| 481 | agtacagtgt ggctgtcaag tgtgccacca tcacccctga |
| | tgaggcccgt gtggaagagt |
| 541 | tcaagctgaa gaagatgtgg aaaagtccca atggaactat |
| | ccggaacatc ctggggggga |
| 601 | ctgtcttccg ggagcccatc atctgcaaaa acatcccacg |
| | cctagtccct ggctggacca |
| 661 | agcccatcac cattggcagg cacgcccatg gcgaccagta |
| | caaggccaca gactttgtgg |
| 721 | cagaccgggc cggcactttc aaaatggtct tcaccccaaa |
| | agatggcagt ggtgtcaagg |
| 781 | agtgggaagt gtacaacttc cccgcaggcg gcgtgggcat |
| | gggcatgtac aacaccgacg |
| 841 | agtccatctc aggttttgcg cacagctgct tccagtatgc |
| | catccagaag aaatggccgc |
| 901 | tgtacatgag caccaagaac accatactga aagcctacga |
| | tgggcgtttc aaggacatct |
| 961 | tccaggagat ctttgacaag cactataaga ccgacttcga |
| | caagaataag atctggtatg |
| 1021 | agcaccggct cattgatgac atggtggctc aggtcctcaa |
| | gtcttcgggt ggctttgtgt |
| 1081 | gggcctgcaa gaactatgac ggagatgtgc agtcagacat |
| | cctggcccag ggctttggct |
| 1141 | cccttggcct gatgacgtcc gtcctggtct gccctgatgg |
| | gaagacgatt gaggctgagg |
| 1201 | ccgctcatgg gaccgtcacc cgccactatc gggagcacca |
| | gaagggccgg cccaccagca |
| 1261 | ccaaccccat cgccagcatc tttgcctgga cacgtggcct |
| | ggagcaccgg gggaagctgg |
| 1321 | atgggaacca agacctcatc aggtttgccc agatgctgga |
| | gaaggtgtgc gtggagacgg |
| 1381 | tggagagtgg agccatgacc aaggacctgg cgggctgcat |
| | tcacggcctc agcaatgtga |
| 1441 | agctgaacga gcacttcctg aacaccacgg acttcctcga |
| | caccatcaag agcaacctgg |
| 1501 | acagagccct gggcaggcag tagggggagg cgccacccat |
| | ggctgcagtg gaggggccag |
| 1561 | ggctgagccg gcgggtcctc ctgagcgcgg cagagggtga |
| | gcctcacagc ccctctctgg |
| 1621 | aggcctttct aggggatgtt tttttataag ccagatgttt |
| | ttaaaagcat atgtgtgttt |
| 1681 | cccctcatgg tgacgtgagg caggagcagt gcgttttacc |
| | tcagccagtc agtatgtttt |
| 1741 | gcatactgta atttatattg cccttggaac acatggtgcc |
| | atatttagct actaaaaagc |
| 1801 | tcttcacaaa aaaaaaaa |

By “Pyruvate kinase M2 (PKM2), polypeptide” is meant a polypeptide or fragment thereof having at least about 85%, or greater, amino acid identity to NCBI Accession No. NP_872271 (below) and having dephosphorylation activity (catalyzing dephosphorylation of phosphoenolpyruvate to pyruvate) and/or the ability to dimerize or tetramerize.

| | |
|---|---|
| 1 | mskphseagt afiqtqqlha amadtflehm crldidsppi |
| | tarntgiict igpasrsvet |
| 61 | lkemiksgmn varlnfshgt heyhaetikn vrtatesfas |
| | dpilyrpvav aldtkgpeir |
| 121 | tglikgsgta evelkkgatl kitldnayme kcdenilwld |
| | yknickvvev gskiyvddgl |
| 181 | islqvkqkga dflvteveng gslgskkgvn lpgaavdlpa |
| | vsekdiqdlk fgveqdvdmv |
| 241 | fasfirkasd vhevrkvlge kgknikiisk ienhegvrrf |
| | deileasdgi mvargdlgie |
| 301 | ipaekvflaq kmmigrcnra gkpvicatqm lesmikkprp |
| | traegsdvan avldgadcim |
| 361 | lsgetakgdy pleavrmghl iareaeaamf hrklfeelvr |
| | asshstdlme amamgsveas |
| 421 | ykclaaaliv ltesgrsahq varyrprapi iavtrnpqta |
| | rqahlyrgif pvlckdpvqe |
| 481 | awaedvdlrv nfamnvgkar gffkkgdvvi vltgwrpgsg |
| | ftntmrvvpv p |

By “PKM2 nucleic acid molecule” is meant a polynucleotide encoding a PKM2 polypeptide. An exemplary PKM2 nucleic acid molecule sequence is provided at NCBI Accession No. NM_182471 (below):

| | |
|---|---|
| 1 | aacccataaa tctgggccct gcccaggtag gccgggacag |
| | ctggggtggc ctgggccgag |
| 61 | agccaagaaa agacacccca tctggcagcc caacttggcg |
| | gcaacaggtg gcccggcgcc |
| 121 | cgggggtctg ggaggaaagt cgctccgggg gcgggccccg |
| | ttgccccgcc gcgtccccat |
| 181 | tggtcatcag gtttcttaaa atgtgactct gaatctgtgt |
| | ccttccgccg cagaatttag |
| 241 | tcccaccgaa agggcaacct gcccgcgcgt tccgccaccg |
| | ccgccgcgct tcctcctgaa |
| 301 | ggtgactgcg cccgcgggga cgcagggggc ggggcccggg |
| | tcgcccggag ccgggattgg |
| 361 | gcagagggcg gggcggcgga gggattgcgg cggcccgcag |
| | cgggataacc ttgaggctga |
| 421 | ggcagtggct ccttgcacag cagctgcacg cgccgtggct |
| | ccggatctct tcgtctttgc |
| 481 | agcgtagccc gagtcggtca gcgccggagg acctcagcag |
| | ccatgtcgaa gccccatagt |
| 541 | gaagccggga ctgccttcat tcagacccag cagctgcacg |
| | cagccatggc tgacacattc |
| 601 | ctggagcaca tgtgccgcct ggacattgat tcaccaccca |
| | tcacagcccg gaacactggc |
| 661 | atcatctgta ccattggccc agcttcccga tcagtggaga |
| | cgttgaagga gatgattaag |
| 721 | tctggaatga atgtggctcg tctgaacttc tctcatggaa |
| | ctcatgagta ccatgcggag |
| 781 | accatcaaga atgtgcgcac agccacggaa agctttgctt |
| | ctgaccccat cctctaccgg |
| 841 | cccgttgctg tggctctaga cactaaagga cctgagatcc |
| | gaactgggct catcaagggc |
| 901 | agcggcactg cagaggtgga gctgaagaag ggagccactc |
| | tcaaaatcac gctggataac |
| 961 | gcctacatgg aaaagtgtga cgagaacatc ctgtggctgg |
| | actacaagaa catctgcaag |
| 1021 | gtggtggaag tgggcagcaa gatctacgtg gatgatgggc |
| | ttatttctct ccaggtgaag |
| 1081 | cagaaaggtg ccgacttcct ggtgacggag gtggaaaatg |
| | gtggctcctt gggcagcaag |
| 1141 | aagggtgtga accttcctgg ggctgctgtg gacttgcctg |
| | ctgtgtcgga gaaggacatc |
| 1201 | caggatctga agtttggggt cgagcaggat gttgatatgg |
| | tgtttgcgtc attcatccgc |
| 1261 | aaggcatctg atgtccatga agttaggaag gtcctgggag |
| | agaagggaaa gaacatcaag |
| 1321 | attatcagca aaatcgagaa tcatgagggg gttcggaggt |
| | ttgatgaaat cctggaggcc |
| 1381 | agtgatggga tcatggtggc tcgtggtgat ctaggcattg |
| | agattcctgc agagaaggtc |
| 1441 | ttccttgctc agaagatgat gattggacgg tgcaaccgag |
| | ctgggaagcc tgtcatctgt |
| 1501 | gctactcaga tgctggagag catgatcaag aagccccgcc |
| | ccactcgggc tgaaggcagt |
| 1561 | gatgtggcca atgcagtcct ggatggagcc gactgcatca |
| | tgctgtctgg agaaacagcc |
| 1621 | aaaggggact atcctctgga ggctgtgcgc atgcagcacc |
| | tgatagctcg tgaggctgag |
| 1681 | gcagccatgt tccaccgcaa gctgtttgaa gaacttgtgc |
| | gagcctcaag tcactccaca |
| 1741 | gacctcatgg aagccatggc catgggcagc gtggaggctt |
| | cttataagtg tttagcagca |
| 1801 | gctttgatag ttctgacgga gtctggcagg tctgctcacc |
| | aggtggccag ataccgccca |
| 1861 | cgtgccccca tcattgctgt gacccggaat ccccagacag |
| | ctcgtcaggc ccacctgtac |
| 1921 | cgtggcatct tccctgtgct gtgcaaggac ccagtccagg |
| | aggcctgggc tgaggacgtg |
| 1981 | gacctccggg tgaactttgc catgaatgtt ggcaaggccc |
| | gaggcttctt caagaaggga |
| 2041 | gatgtggtca ttgtgctgac cggatggcgc cctggctccg |
| | gcttcaccaa caccatgcgt |
| 2101 | gttgttcctg tgccgtgatg gaccccagag cccctcctcc |
| | agcccctgtc ccaccccctt |
| 2161 | cccccagccc atccattagg ccagcaacgc ttgtagaact |
| | cactctgggc tgtaacgtgg |
| 2221 | cactggtagg ttgggacacc agggaagaag atcaacgcct |
| | cactgaaaca tggctgtgtt |
| 2281 | tgcagcctgc tctagtggga cagcccagag cctggctgcc |
| | catcatgtgg ccccacccaa |
| 2341 | tcaagggaag aaggaggaat gctggactgg aggcccctgg |
| | agccagatgg caagagggtg |
| 2401 | acagcttcct ttcctgtgtg tactctgtcc agttccttta |
| | gaaaaaatgg atgcccagag |
| 2461 | gactcccaac cctggcttgg ggtcaagaaa cagccagcaa |
| | gagttagggg ccttagggca |
| 2521 | ctgggctgtt gttccattga agccgactct ggccctggcc |
| | cttacttgct tctctagctc |
| 2581 | tctaggcctc tccagtttgc acctgtcccc accctccact |
| | cagctgtcct gcagcaaaca |
| 2641 | ctccaccctc caccttccat tttcccccac tactgcagca |
| | cctccaggcc tgttgctata |
| 2701 | gagcctacct gtatgtcaat aaacaacagc tgaagcacca |
| | aaaaaaaaaa aaaa |

By “PKM2 Inhibitor” is meant an agent that inhibits PKM2 expression, function or activity. Exemplary PKM2 inhibitors are known in the art and described, for example, by Heiden et al., Biochem Pharmacol. 2010 Apr. 15; 79(8): 1118-1124 and Dong et al., Oncol Lett. 2016 March; 11(3): 1980-1986. PKM2 inhibitors include, but are not limited to, PKM2 inhibitor compound 3k (Selleckchem), DASA-58 (Selleckchem), and PKM2 inhibitory nucleic acids.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease, such as cancer.

By “alteration” is meant a change (increase or decrease) in the expression levels or activity of a gene or polypeptide as detected by standard art known methods such as those described herein. As used herein, an alteration includes a 10% change in expression or activity levels, preferably a 25% change, more preferably a 40% change, and most preferably a 50% or greater change in expression levels. In one embodiment, an increase in Skp2, IDH1, or IDH2 expression is at least 5, 10, 15, 20, 25% or more relative to a reference cell at a corresponding stage of the cell cycle.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing,” and “having” and the like can have the meaning ascribed to them in U.S. patent law, and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. In a disease, such as cancer (e.g., breast cancer, prostate cancer, glioblastoma).

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount of an agent defined herein is sufficient to reduce or stabilize the proliferation of a cancer cell. In another embodiment, an effective amount of an agent defined herein is sufficient to kill a cancer cell.

The invention provides a number of targets that are useful for the development of highly specific drugs to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Exemplary markers include Skp2, p27, p21, Cyclin A, Cyclin E, IDH1, IDH2, glycolysis, TCA cycle, lactate levels, oxidative phosphorylation levels.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D show that cells in S phase or G1 phase rely on glycolysis or TCA cycle, respectively. FIG. 1A is a schematic illustration of the experimental procedure for studies performed in FIGS. 1B and 1D. HeLa cells were arrested in mitosis by 10 μg/mL nocodazole blockage for 20 hours and the mitotic cells were shaken off, washed twice with PBS and re-plated for the indicated times, followed by extracellular acidification rate (ECAR) and oxygen consumption rate (OCR) measurements with Seahorse XF24 extracellular flux analyzer. FIG. 1B is a graph depicting ECAR results of cells after being synchronized and released at the indicated times to illustrate cell cycle-dependent fluctuation of glycolysis usage. The concentration of glucose, oligomycin and 2-DG are 10 mM, 1 μM and 50 mM, respectively (mean±s.e.m, n=4). FIG. 1C depicts flow cytometry analysis for cells after release from nocodazole blockage to indicate the cell cycle profile at each collected time point. HeLa cells were synchronized by nocodazole blockage for 20 hours and released at the indicated times. Cells were then trypsinized, fixed with 75% ethanol, stained with propidium iodide (PI), and subjected to flow cytometry analysis. FIG. 1D is a graph depicting OCR results of cells after being synchronized and released at the indicated times to illustrate cell cycle-dependent fluctuation of TCA cycle usage. The concentrations of oligomycin, FCCP and antimycin A are 1 μM, 0.3 μM and 1 μM, respectively (mean±s.e.m, n=4).

FIGS. 2A-2G show that cells in S phase have higher glycolytic flux and relatively lower TCA cycle flux than cells in G1 phase. FIG. 2A depicts flow cytometry analysis for cells after being released from nocodazole blockage for the indicated times to indicate the cell cycle profile at each collected time point. HeLa cells were synchronized by 10 μg/mL nocodazole blockage for 20 hours and released at the indicated times. Cells were then trypsinized, fixed with 75% ethanol, stained with PI and subjected to flow cytometry analysis. FIG. 2B is a schematic illustration of the experimental procedure for studies performed in FIGS. 15C, 15D, 2C, and 2E. HeLa cells were arrested in mitosis by 10 μg/mL nocodazole blockage for 20 hours and the mitotic cells were shaken off, washed twice with PBS and re-plated for the indicated times, followed by either U-¹³C6-glucose (0, 30, 60 and 120 s) or U-¹³C5-glutamine labeling (0, 1, 2 and 3 hr). FIG. 2C depicts graphs showing that cells synchronized in S phase have higher glycolytic flux than those in G1 phase. Intracellular accumulation of ¹³C-labeled glycolytic intermediates (F6P, fructose-6-phosphate; FBP, fructose-1,6-bisphosphate; GA3P, glyceraldhyde-3-phosphate; 3PG, 3-phosphoglycerate; PEP, phosphoenolpyruvate) after switching 1×10⁶ into uniformly ¹³C-labeled glucose media for either 0, 30, 60 or 120 seconds (mean±s.e.m, n=3). FIG. 2D shows that differences in glycolytic flux between cells in S phase and cells in G1 phase appear independent of glucose uptake. FIG. 2E depicts graphs showing that cells synchronized in S phase have lower TCA flux than those in G1 phase. Intracellular accumulation of ¹³C-labeled TCA cycle intermediates after switching 1×10⁶ into uniformly ¹³C-labeled glutamine media for either 0, 1, 2 or 3 hours (mean±s.e.m, n=3). FIG. 2F shows that differences in TCA flux between S phase cells and G1 phase cells appear not to be dependent on glutamine uptake. FIG. 2G depicts graphs showing that cells synchronized in S phase have higher flux in pentose phosphate pathway (PPP) than those in G1 phase. Intracellular accumulation of ¹³C-labeled PPP intermediates (6PGL, 6-phosphogluconolactone; R5P, ribose-5-phosphate; S7P, sedoheptulose-7-phosphate) of cells as in FIG. 2C (mean±s.e.m, n=3).

FIGS. 3A-3D show that IDH1/IDH2 expression fluctuates during cell cycle. FIG. 3A is a schematic diagram showing the TCA cycle enzymes that were monitored for cell cycle-dependent expression pattern in this study. FIG. 3B depicts flow cytometry analysis of HeLa cells after released from nocodazole blockage for indicated time to indicate cell cycle profile at each collected time point. FIG. 3C depicts immunoblots showing that IDH1 and IDH2 protein abundance fluctuates during cell cycle progression in HCT116 cells. HCT116 cells were synchronized and released at the indicated times, followed by immunoblot of the indicated proteins. FIG. 3D is a schematic diagram illustrating the observed fluctuation of IDH1/2 protein levels that correlate with the metabolic oscillation of glycolysis and TCA cycle during the cell cycle.

FIGS. 4A-4J show that both IDH1 and IDH2 play important roles in governing TCA cycle, and depletion of either isoform results in comparable changes in metabolic phenotypes. FIG. 4A is a graph depicting OCR analysis of WT cells in comparison with HAP1-IDH1^{-/-} and IDH2^{-/-} cells. HAP1-IDH1^{-/-} and IDH2^{-/-} cells were made by CRISPR/Cas 9-mediated depletion of IDH1 or IDH2 (mean±s.e.m, n=6). FIG. 4B is a graph depicting ECAR analysis of WT cells in comparison with HAP1-IDH1^{-/-} and IDH2^{-/-} cells (mean±s.e.m, n=6). FIG. 4C depicts graphs showing intracellular accumulation of ¹³C-labeled TCA cycle intermediates in WT versus IDH1^{-/-} and IDH2^{-/-} cells after switching into uniformly ¹³C-labeled glutamine media for either 0, 1 or 2 hours (mean±s.e.m, n=3). FIG. 4D shows that loss of either IDH1 or IDH2 impairs oxidative phosphorylation in HAP1 cells. Ten thousands of HAP1-WT, IDH1^{-/-} or IDH2^{-/-} cells were cultured in DMEM media with either glucose or galactose for 6 days, and the growth curve was drawn. FIG. 4E is an immunoblot of IDH1 in HeLa-IDH1^{+/+} and IDH1^{-/-}. HeLa-IDH1^{-/-} cells were made using CRISPR/Cas 9. FIGS. 4F and 4G are graphs depicting ECAR results of IDH1^{+/+} and IDH1^{-/-} HeLa cells. The concentration of glucose, oligomycin and 2-DG are 10 mM, 1 μM and 50 mM, respectively (mean±s.e.m, n=3, *** p<0.001). FIGS. 4H and 4I are graphs depicting OCR results of HeLa-IDH1^{+/+} and IDH1^{-/-} cells. The concentration of oligomycin, FCCP and antimycin A are 1 μM, 0.3 μM and 1 μM, respectively (mean±s.e.m, n=4, *** p<0.001). FIG. 4J is a graph showing that depletion of IDH1 leads to more lactate release in HeLa cells. The extracellular lactate was measured in IDH1^{+/+} and IDH1^{-/-} HeLa cells at the indicated times, n=3, ** p<0.01, *** p<0.001.

FIGS. 5A-5K show that Skp2 is an upstream E3 ubiquitin ligase for IDH1. FIG. 5A depicts immunoblots showing that IDH2 specifically interacts with Cullin 1 in cells. Immunoblot analysis of immunoprecipitates (IP) and whole cell lysates (WCL) derived from HEK293 cells transfected with Flag-IDH2 and Myc-tagged Cullins for 48 hours. FIG. 5B depicts immunoblots showing that knockdown of Cullin 1, but not Cullin 3, leads to accumulation of IDH1 in cells. PC3 cells were infected with shControl, shCulin1 or shCullin 3 lenti-viruses, selected for 3 days, followed by immunoblot analysis for indicated proteins. FIG. 5C depicts immunoblots showing that depletion of Cullin 4A or Cullin 4B does not affect IDH1 levels in MEFs. Immunoblot of WCL derived from Cullin 4A^{+/+}, Cullin 4A^{-/-}, Cullin 4B^{+/+} and Cullin 4B^{-/-} MEFs. FIG. 5D depicts immunoblots showing that IDH1 interacts with the essential SCF component, Skp1 in cells. Immunoblots of IP and WCL derived from HEK293 cells transfected with Flag-IDH1 and Myc-Skp1. FIG. 5E depicts immunoblots showing that IDH1 interacts with the essential SCF component, Rbx1 in cells. Immunoblots were performed on IP and WCL derived from HEK293 cells transfected with Flag-IDH1 and HA-Rbx1. FIG. 5F depicts immunoblots showing that IDH2 interacts with Skp1 in cells. Immunoblots of IP and WCL derived from HEK293 cells transfected with Flag-IDH2 and Myc-Skp1. FIG. 5G depicts immunoblots showing that IDH2 interacts with Rbx1 in cells. Immunoblots were performed on IP and WCL derived from HEK293 cells transfected with Flag-IDH2 and HA-Rbx1. FIGS. 5H and 5I depict immunoblots showing that knockdown of Skp2 leads to accumulation of IDH1 in HCT116 (FIG. 5H) or DLD1 (FIG. 5I) cells. HCT116 and DLD1cells were infected with shControl or shSkp2 virus, selected for 3 days, followed by immunoblot analysis for indicated proteins. FIGS. 5J-5K depict immunoblots showing that knockdown of Fbw4 fails to accumulate IDH1 in HeLa (FIG. 5J) and U2OS (FIG. 5K) cells. HeLa and U2OS cells were infected with shControl or shFbw4 lenti-viruses, selected for 3 days, followed by immunoblot analysis for the indicated proteins.

FIGS. 6A-6G show that knockdown of Skp2 changes the metabolic phenotype of the resulting cells during cell cycle progression. FIGS. 6A and 6B are graphs depicting ECAR results of shControl (FIG. 6A) or shSkp2 (FIG. 6B) expressing HeLa cells after synchronization and release for the indicated times. HeLa cells were infected with shControl or shSkp2 lenti-viruses, selected with puromycin for 3 days and then subjected to synchronization and Seahorse analysis. The concentration of glucose, oligomycin and 2-DG are 10 mM, 1 μM and 50 mM (mean±s.e.m, n=4), respectively. FIGS. 6C and 6D are graphs showing OCR results of shControl (FIG. 6C) or shSkp2 (FIG. 6D) expressing HeLa cells after being synchronized and released at the indicated times. Cells are the same as in FIGS. 6A-6B. The concentration of oligomycin, FCCP and antimycin A are 1 μM, 0.3 μM and 1 μM (mean±s.e.m, n=4), respectively. FIG. 6E depicts flow cytometry of shControl or shSkp2 expressing HeLa cells after synchronization and release for the indicated times. Cells were then trypsinized, fixed with 75% ethanol, stained with propidium iodide and subjected to flow cytometry analysis. FIG. 6F is a graph depicting knockdown of Skp2 eliminates lactate release at S phase. HeLa cells were infected with shControl and shSkp2 lenti-viruses, selected for 3 days, synchronized by nocodazole blockage and released at the indicated times, followed by measurement of lactate release in the media. FIG. 6G is a graph showing that knockdown of Skp2 eliminates lactate release at S phase. HeLa cells were infected with shControl and shSkp2 lenti-viruses, selected for 3 days, synchronized by double thymidine blockage and released at the indicated times, followed by measurement of lactate release in the media.

FIGS. 7A-7Q show that the IDH1-T157A mutant is resistant to Skp2-mediated degradation to confer metabolic phenotype change during cell cycle. FIG. 7A depicts immunoblots showing that Skp2 promotes degradation of IDH2 in a cyclin E/CDK2-dependent manner. Immunoblot analysis of WCL derived from HEK293 cells that were transfected with Flag-IDH2 and indicated constructs. FIG. 7B depicts the conserved TP/SP sites within IDH1 and IDH2 protein sequence among different species. FIG. 7C depicts immunoblots showing depletion of CCNE1/2 leads to the accumulation of IDH1 and IDH2. Immortalized CCNE1^{-/-}E2^{-/-} MEFs were harvested for immunoblot of the indicated proteins. FIG. 7D depicts immunoblots showing depletion of CCNA2 leads to the accumulation of IDH1 and IDH2. WT and CCNA2^{-/-} MEFs were infected with Cre lenti-virus and subjected to IB for indicated proteins. FIG. 7E depicts immunoblots showing depletion of CCND does not lead to the accumulation of IDH1 and IDH2. CCND1^{-/-}, CCND2^{-/-}, CCND3^{-/-} and WT MEFs were harvested for D3 of indicated proteins. FIGS. 7F-7G are graphs showing depletion of CCNE1 leads to elevation of OCR. CCNE1^{-/-}, CCNE2^{-/-} and WT MEFs were subjected to OCR measurement using Seahorse XF extracellular flux analyzer, *** p<0.001. FIG. 7H is a mass spectrum for the phosphorylation of IDH1 on T157 residue. HEK293 cells were co-transfected with Flag-IDH1, HA-cyclin E and HA-CDK2, followed by anti-Flag-IDH1 IP and SDS-PAGE. The band containing IDH1 was collected for subsequent LC-MS/MS. FIG. 7I depicts sequences of synthetic peptides. FIG. 7J is an immunoblot showing that Skp2 recognizes synthetic phosphorylated peptides of IDH1 (T157) and IDH2 (T197), but not non-phosphorylated peptides in vitro. 2 μg peptide was incubated with 10 μg recombinant GST-Skp2 proteins for 4 hours, and pulled down by 10 μl Streptavidin agarose, followed by SDS-PAGE for immunoblot of GST. FIG. 7K is an immunoblot showing that Skp2, but not Fbw4, binds to synthetic phosphorylated peptides of IDH1 (T157) in vitro. 2 μg peptide was incubated with 10 μg recombinant GST-Skp2 or GST-Fbw4 proteins for 4 hours, and pulled down by 10 μl Streptavidin agarose, followed by SDS-PAGE for immunoblot of GST. FIGS. 7L and 7M depict immunoblots of the indicated proteins in HeLa (FIG. 7L) and U2OS (FIG. 7M) cells stably expressing HA-IDH1-WT or indicated mutants, with or without knocking down Skp2. Cells were infected with HA-IDH1-WT, HA-IDH1-T157A, or HA-IDH1-T77A lenti-virus, selected with hygromycin B for 3 days, then infected with either shControl or shSkp2 virus, selected with puromycin for 3 days, followed by immunoblot assay. FIGS. 7N and 7O are graphs depicting ECAR results of IDH1-WT (FIG. 7N) or IDH1-T157A (FIG. 7O) expressing HeLa cells after synchronized and released at the indicated times. HeLa cells were infected with IDH1-WT or IDH1-T157A virus, selected with hygromycin B for 3 days and then subjected to synchronization and Seahorse analysis. The concentration of glucose, oligomycin and 2-DG are 10 mM, 1 μM and 50 mM (mean±s.e.m, n=4), respectively. FIGS. 7P and 7Q are graphs showing OCR results of IDH1-WT (FIG. 7P) or IDH1-T157A (FIG. 7Q) expressing HeLa cells after synchronized and released at the indicated times. Cells were generated and processed as described in FIGS. 17I-17J. The concentration of oligomycin, FCCP and antimycin A are 1 μM, 0.3 μM and 1 μM (mean±s.e.m, n=4), respectively.

FIGS. 8A-8I show that, compared to expressing IDH1-WT, ectopic expression of IDH1-T157A leads to higher cell population in G1 phase, which retards cell proliferation and clonal formation. FIG. 8A depicts flow cytometry results showing that cells expressing an IDH1-T157A mutant have a high population of cells in G1 phase compared to cells expressing IDH1-WT. FIG. 8B is an immunoblot of U2OS cell lines stably expressing IDH1-WT or the indicated IDH1 mutants. FIG. 8C is a growth curve of U2OS cell lines stably expressing IDH1-WT or the indicated IDH1 mutants. FIG. 8D depicts colony formation assays for U2OS cells stably expressing IDH1-WT or the indicated IDH1 mutants. FIG. 8E is a graph depicting quantification of colony formation results derived from FIG. 8D (mean±s.e.m, n=3, * p<0.05). FIG. 8F is an immunoblot of T98G cell lines stably expressing IDH1-WT or the indicated IDH1 mutants. FIG. 8G depicts growth curves of T98G cell lines stably expressing IDH1-WT or IDH1mutants. FIG. 8H depicts colony formation assays for T98G cells stably expressing IDH1-WT or mutants. FIG. 8I is a graph depicting quantification of colony formation results derived from FIG. 8H (mean±s.e.m, n=3, * p<0.05).

FIGS. 9A-9D show that cyclin E/CDK2 recognizes IDH1 through a conserved RGL motif. FIG. 9A is a schematic illustration of the conserved RXL motif in proteins that bind cyclin E. FIG. 9B are immunoblots showing that tumor derived IDH1 mutant, R338T abolishes its binding with cyclin E. Immunoblot analysis of IP and WCL derived from HEK293 cells that were transfected with cyclin E with either IDH1-WT or IDH1-R338T mutation. FIG. 9C are immunoblots showing that the R338T mutant escapes from Skp2-mediated ubiquitination in cells. Immunoblot analysis was preformed on Ni-NTA pull down products and WCL derived from HEK293 cells that were transfected with His-Ub and IDH1 constructs. FIG. 9D is a schematic illustration of a working model for Skp2-mediated ubiquitination and subsequent degradation of IDH1 that requires prior phosphorylation of IDH1 at the Thr157 residue by the CDK2/Cyclin E kinase to trigger its interaction by Skp2. In S phase, cyclin E/CDK2 binds IDH1 through an RGL motif in IDH1, and phosphorylates the latter one at Thr157 residue. After phosphorylation, IDH1 can be recognized and subsequently ubiquitinated by SCF^{Skp2}, and eventually degraded by 26S proteasome.

FIGS. 10A-10F show that Skp2 and IDH1 protein abundance inversely correlate in a panel of prostate cancer (PrCa) cells. FIG. 10A depict immunoblots showing that different Akt activation levels in PrCa cells are not correlated with IDH1/2 protein abundance. FIG. 10B ECAR analysis of different PrCa cells. Cells were plated into XF24 plate 48 hours prior to the measurement (10,000 for DU145 and PC3; 20, 000 for C4-2, LNCaP and RV1). After measurement, cell number was counted and results were normalized to cell number. The concentration of glucose, oligomycin and 2-DG are 10 mM, 1 μM and 50 mM, respectively (mean±s.e.m, n=4). FIG. 10C is a graph depicting OCR analysis of different PrCa cells. The concentration of oligomycin, FCCP and antimycin A are 1 μM, 0.3 μM and 1 μM, respectively (mean±s.e.m, n=4). FIG. 10D provides a summary of protein abundance of Skp2 and IDH1, as well as their correlation with different metabolic phenotypes measured by OCR, indicative of TCA cycle rate, or ECAR, indicative of glycolysis rate, in different PrCa cells. FIG. 10E is a graph showing ECAR analysis of Skp2^{high} PrCa cells with or without depleting endogenous Skp2. Skp2^{high} PrCa cells, including DU145 and PC3, were infected with shControl or shSkp2 virus, selected with puromycin for 5 days, and subjected to Seahorse analysis. Cells were plated into XF24 plate 48 hours prior the measurement (10,000 for shControl, 20, 000 for shSkp2). After measurement, cell number was counted and results were normalized to cell number. The concentration of glucose, oligomycin and 2-DG are 10 mM, 1 μM and 50 mM (mean±s.e.m, n=4), respectively. FIG. 10F is a graph showing OCR analysis of Skp2^{high} PrCa cells with or without knocking down endogenous Skp2. Cells were the same as in FIG. 10E. The concentration of oligomycin, FCCP and antimycin A are 1 μM, 0.3 μM and 1 μM, respectively (mean±s.e.m, n=4).

FIGS. 11A-11H show that inhibiting Skp2 with a specific inhibitor, SKPin C1, changes the metabolic phenotype of PrCa cells. FIG. 11A are immunoblots showing that SKPin C1 treatment leads to accumulated protein abundance of IDH1 and IDH2 in LNCaP cells. LNCaP cells were treated with 0, 1, 3, 10 or 30 μM SKPin C1 for 24 hours, harvested for immunoblot analysis of indicated proteins. FIG. 11B depicts immunoblots showing that SKPin C1 treatment leads to accumulated protein abundance of IDH1 and IDH2 in cytoplasm. 22Rv1 cells were treated with 10 μM SKPin C1 for 24 hours, harvested for fraction of cytoplasm (C), mitochondria (M), and nuclei (N), followed by immunoblot analysis of indicated proteins. FIG. 11C is a graph showing depletion of endogenous ID111 or IDH2 abolishes the effect of SKPin C1 on OCR. HAP1-IDH1^{-/-}, IDH2^{-/-} and parental cells were treated with 3 μM SKPin C1 for 24 hours, followed by OCR analysis by Seahorse XF 24 analyzer. The concentration of oligomycin, FCCP and antimycin A are 1 μM, 0.6 μM and 3 μM, respectively (mean±s.e.m, n=3). FIG. 11D depicts immunoblots showing depletion of Skp2 abolishes the effect of SKPin C1 on IDH1 and IDH2. HAP1-Skp2^{+/+} and Skp2^{-/-} cells were treated with indicated SKPin C1 for 24 hours, followed by immunoblot analysis for indicated proteins. FIG. 11E is a graph showing depletion of Skp2 abolishes the effect of SKPin C1 on ECAR. Statistical analysis, ** p<0.01. FIG. 11F is a graph showing that depletion of Skp2 abolishes the effect of SKPin C1 on OCR. Statistical analysis, * p<0.05, ** p<0.01. FIG. 11G is a graph showing that depletion of Skp2 abolishes the effect of SKPin C1 on ECAR. HAP1-Skp2^{+/+} and Skp2^{-/-} cells were treated with 1 μM SKPin C1 for 24 hours, followed by ECAR analysis by Seahorse XF 24 analyzer. FIG. 11H is a graph showing that depletion of Skp2 abolishes the effect of SKPin C1 on OCR. HAP1-Skp2^{+/+} and Skp2^{-/-} cells were treated with 1 μM SKPin C1 for 24 hours, followed by OCR analysis by Seahorse XF 24 analyzer. The concentration of oligomycin, FCCP and antimycin A are 1 μM, 0.6 μM and 3 μM, respectively (mean±s.e.m, n=3, ** p<0.01, *** p<0.001).

FIGS. 12A-12H show that Skp2 governs IDH1 protein stability and metabolic oscillation in cell cycle independently of p27. FIGS. 12A and 12B are immunoblots showing that depletion of endogenous p27 in HeLa cells (FIG. 12A) and HCT116 cells (FIG. 12B) has minimal effect on IDH1 or IDH2 abundance in cells. Cells were infected with shControl or shp27 virus, selected for 3 days, followed by immunoblot analysis for the indicated proteins.

FIGS. 12C and 12D are graphs showing that depletion of p27 has minimal effect on ECAR in HeLa cells. The concentration of glucose, oligomycin and 2-DG are 10 mM, 1 μM and 50 mM, respectively (mean±s.e.m, n=4). FIGS. 12E and 12F are graphs showing that depletion of endogenous p27 has minimal effect on OCR in HeLa cells. The concentration of oligomycin, FCCP and antimycin A are 1 μM, 0.3 μM and 1 μM, respectively (mean±s.e.m, n=4). FIG. 12G is a schematic diagram illustrating that Skp2 regulates cell cycle progression and metabolic oscillation through governing the protein stability of its substrates p27 and IDH1, respectively. FIG. 12H is a schematic diagram illustrating the fluctuation of Skp2/cyclin E/IDH1 levels and the corresponding metabolic glycolysis/TCA cycle during cell cycle progression.

FIGS. 13A-13G Depletion of IDH1 redirect the changes in cell metabolism caused by depleting Skp2. FIG. 13A depicts immunoblots of HeLa cells infected with either shControl, shSkp2, or shSkp2+shIDH1 lenti-viruses, selected for 3 days, arrested in mitosis by 10 μg/mL nocodazole blockage for 20 hours, released for either 6 (G1 phase) or 12 hours (S phase), followed by immunoblot analysis for the indicated proteins. FIGS. 13B and 13 C are graphs showing ECAR measurements of cells in FIG. 13A. FIGS. 13D and 13E are graphs showing OCR measurements of cells in FIG. 13A. FIG. 13F depicts colony formation assays for the indicated HeLa cells. HeLa cells infected with either shControl, shSkp2, or shSkp2+shIDH1 lenti-viruses, plated in 6-well plate (300 cell/well) for 3 weeks. FIG. 3G is a graph depicting quantification of colony formation results derived from FIG. 13F (mean±s.e.m, n=3, *** p<0.001).

FIG. 14 is a schematic illustration of a working model for Skp2 in controlling cell cycle progress and cell metabolic shift via ubiquiting and subsequent degrading of p27 and IDH1/2, respectively.

FIGS. 15A-15I show that mammalian cells adopt different glucose metabolism pathways in different cell cycle stages, primarily utilizing TCA cycle in G1 phase, but relying on glycolysis in S phase. FIG. 15A is a graph showing that glycolysis, measured as extracellular acidification rate (ECAR), is higher for cells in early S phase than in G1 phase. HeLa cells were synchronized by 10 μg/mL nocodazole blockage for 20 hours and subsequently released at the indicated time points. From the ECAR curve, glycolysis (ECAR level when the present of glucose), glycolytic capacity (stimulated glycolysis when oligomycin is used to inhibit ATP synthase), and glycolytic reserve (glycolytic capacity minus glycolysis) were determined. n=4, * p<0.05, ** p<0.01, *** p<0.001. FIG. 15B is a graph showing that TCA cycle, measured as oxygen consumption rate (OCR), is lower for cells in early S phase than in G1 phase. HeLa cells were synchronized by 10 μg/mL nocodazole blockage for 20 hours and subsequently released at the indicated time points. From the OCR curve, basal respiration, ATP production (the OCR portion that is inhibited by oligomycin), and maximal respiration (stimulated OCR when antimycin A is used to inhibit electron transfer chain complex III) were determined. n=4, * p<0.05, ** p<0.01. FIG. 15C is a graph showing that glycolytic flux is higher for cells in S phase than in G1 phase. HeLa cells were synchronized with nocodazole blockage and released for 6 hours (G1 phase) or 12 hours (S phase), labeled with ¹³C-glucose for 1 minute, followed by measuring for labeled glycolytic intermediates. n=3, * p<0.05. FIG. 15D is a graph showing that TCA cycle flux is lower for cells in S phase than in G1 phase. HeLa cells were synchronized with nocodazole and released for 6 hours (G1 phase) or 12 hours (S phase), labeled with ¹³C glutamine for 1 hour, followed by measuring the labeled TCA cycle intermediates. n=3, * p<0.05, ** p<0.01, *** p<0.001. FIG. 15E are immunoblots showing that protein abundance of IDH1, and to a lesser extent of IDH2, fluctuates during the cell cycle. HeLa cells were synchronized by 10 μg/mL nocodazole blockage for 20 hours and subsequently released at the indicated time points before harvesting for immunoblot (IB) analysis. FIG. 15F is a graph showing that depletion of IDH1 or IDH2 leads to slight increase of glycolysis (ECAR). HAP1-IDH1^{-/-} and HAP1-IDH2^{-/-} cells were generated in HAP1 cells using CRISPR/Cas9. The ECAR of HAP1-IDH HAP1-IDH2^{-/-} and parental cells (WT) were measured using Seahorse XF24 analyzer. * p<0.05, ** p<0.01. FIG. 15G is a graph showing that depletion of IDH1 or IDH2 compromises OCR. * p<0.05, ** p<0.01. FIG. 15H is a graph showing that depletion of IDH1 or IDH2 compromises TCA cycle flux. HAP1-IDH^{-/-}, HAP1-IDH2^{-/-} and parental cells (WT) were labeled with ¹³C glutamine for 1 hour followed by measuring the labeled TCA cycle intermediates. * p<0.05, ** p<0.01. FIG. 15I depicts immunoblots of IDH1 and IDH2 in HAP1-IDH^{-/-} and HAP1-IDH2^{-/-} cells.

FIGS. 16A-16L show that SCF^{Skp2} promotes IDH1 ubiquitination and subsequent degradation to trigger the timely switch to glycolysis in the S phase. FIG. 16A are immunoblots showing that MG132 and MLN4924 treatment leads to accumulation of IDH1 and IDH2. RWPE1 cells were incubated with 10 μM MG132 or MLN4924 for 8 hours, followed by immunoblot analysis of IDH1 and IDH2. FIG. 16B are immunoblots showing that IDH1 specifically interacts with Cullin 1 in cells. Immunoblot analysis of immunoprecipitates (IP) and whole cell lysates (WCL) derived from HEK293 cells transfected with Flag-IDH1 and the indicated Myc-tagged Cullins for 48 hours. FIG. 16C are immunoblots showing that IDH1 specifically interacts with two F-box proteins, Skp2, and to a lesser extent, Fbw4, in cells. Immunoblot analysis of IP and WCL derived from HEK293 cells transfected with Flag-IDH1 and the indicated CMV-GST-tagged F-box proteins for 48 hours. FIG. 16D are immunoblots showing depletion of SKP2 in HeLa cells leads to accumulated IDH1. HeLa cells were infected with pLKO-shSkp2 or mock lenti-viruses, selected with puromycin (1 μg/mL) for 3 days to eliminate non-infected cells, and subjected to D3 analysis with the indicated antibodies. FIG. 16E are immunoblots showing that genetic ablation of Skp2 in mouse embryonic fibroblasts (MEFs) leads to a significant increase in protein abundance of IDH1 and IDH2. Primary Skp2^{+/+} and Skp2^{-/-} MEFs were harvested and subjected to immunoblot analysis with the indicated antibodies. FIG. 16F are immunoblots showing that Skp2 interacts with IDH1 and IDH2 in cells. HEK293 cell lysates were subjected to pull down by anti-Skp2 antibody and protein A/G agarose, followed by immunoblot analysis with the indicated antibodies. FIG. 16G are immunoblots showing that Skp2, but not Fbw4, promotes IDH1 ubiquitination in cells. Immunoblot analysis of Ni-NTA pull down products and WCL derived from HEK293 cells transfected with the indicated constructs. FIG. 16H are immunoblots showing depletion of endogenous SKP2 abolishes IDH1/2 expression fluctuation during the cell cycle. HeLa cells were infected with pLKO-shSkp2 or mock lenti-viruses, and selected with puromycin for 3 days to eliminate non-infected cells. The resulting cells were synchronized by 10 μg/mL nocodazole blockage for 20 hours and subsequently released at the indicated time points. Then cells were harvested and WCL was subjected to immunoblot analysis with the indicated antibodies. FIG. 16I is a graph showing that depletion of endogenous SKP2 abolishes the glycolytic peak in S phase. After releasing for the indicated time points, cell lines generated in FIG. 16H were subjected to ECAR measurement using Seahorse XF extracellular flux analyzer. n=3, * p<0.05, **p<0.01. FIG. 16J is a graph showing that depletion of endogenous SKP2 impairs the decrease of OCR in S phase. After releasing for indicated time, various cell lines generated in FIG. 16H were subjected to OCR measurement using Seahorse XF extracellular flux analyzer. n=3, * p<0.05, ** p<0.01. FIG. 16K is a graph showing that depletion of endogenous SKP2 eliminates the observed difference in glycolytic flux between G1 phase and S phase. Various cell lines generated in FIG. 16H were synchronized and released at the indicated time points, followed by ¹³C-glucose labeling for 60 seconds. The labeled glycolytic intermediates were measured using HPLC-MS. n=3, * p<0.05, ** p<0.01, ***p<0.001. FIG. 16L is a graph showing that depletion of endogenous SKP2 eliminates the observed difference in TCA cycle flux between G1 and S phase. Various cell lines generated in FIG. 16D were synchronized and released at the indicated time points, followed by ¹³C-glutamine labeling for 1 hour. The labeled TCA cycle intermediates were measured using HPLC-MS. n=3, * p<0.05, ** p<0.01, *** p<0.001.

FIGS. 17A-17O show that Cyclin E/CDK2 and/or Cyclin A/CDK2 phosphorylates IDH1 to trigger its ubiquitination and subsequent degradation by SCF^{Skp2}. FIG. 17A are immunoblots showing that Skp2 promotes IDH1 degradation in a cyclin E/CDK2 and/or cyclin A/CDK2-dependent manner in cells. Immunoblot analysis of HeLa cells after transfecting with Flag-IDH1 and indicated constructs for 48 hours. FIG. 17B depicts an immunoblot and SDS-PAGE showing that cyclin E/CDK2 phosphorylates IDH1 in vitro. Bacterially purified GST or GST-IDH1 recombinant proteins were incubated with purified cyclin E/CDK2 for 30 minutes at 30° C. with ³²P-γ-ATP as donor of phosphorylation, followed by SDS-PAGE. The protein input was stained with Coomassie brilliant blue. FIG. 17C depicts an immunoblot and SDS-PAGE showing identification of the T157 residue as the major site being phosphorylated by cyclin E/CDK2 in vitro. Bacterially purified His-IDH1-WT or mutant proteins were incubated with purified cyclin E/CDK2 for 30 minutes with ³²P-γ-ATP as donor of phosphorylation, followed by SDS-PAGE. The protein input was stained with Coomassie brilliant blue. FIG. 17D depict immunoblots showing that genetic ablation of CCNE1^{-/-} but not CCNE2^{-/-} in MEFs leads to a significant increase in protein abundance of IDH1 and IDH2. Immortalized CCNE1^{-/}, ⁻CCNE2^{-/-} and WT MEFs were harvested and subjected to immunoblot analysis with the indicated antibodies. FIG. 17E depicts an immunoblot and SDS-PAGE showing that cyclin E/CDK2-dependent phosphorylation of T157 is required for IDH1 to be recognized by recombinant Skp2 in vitro. Bacterially purified recombinant GST-IDH1-WT or the indicated mutant IDH1 proteins were incubated with or without purified cyclin E/CDK2 for 30 minutes with ATP as donor of phosphorylation, followed by His-Skp2 pull down, and then subjected to SDS-PAGE and immunoblot analysis. The protein input was stained with Coomassie brilliant blue. FIG. 17F depicts immunoblots showing that the IDH1-T157A mutant is impaired to undergo Skp2-dependent ubiquitination in cells, compared to WT-IDH1. Immunoblot analysis of Ni-NTA pull down products and WCL derived from HEK293 cells transfected with Flag-IDH1-WT or IDH1 mutants, together with other indicated constructs. FIG. 17G depicts immunoblots showing that the IDH1-T157A mutant is resistant to Skp2-dependent degradation in cells. Immunoblot analysis of WCL derived from HeLa cells transfected with Flag-IDH1-WT or T157A mutant and other indicated constructs. FIG. 17H depict immunoblots showing that, compared to WT-IDH1, the IDH1-T157A mutant escapes from cell cycle-dependent degradation, thereby becoming stabilized across different cell cycle phases. HeLa cells were infected with HA-IDH1-WT or T157A lenti-viruses and selected with hygromycin B (200 μg/mL) for 3 days. The stable cell lines were synchronized by nocodazole blockage for 20 hours and released at the indicated time points, followed by immunoblot analysis with the indicated antibodies. FIG. 17I is a graph showing that, compared to WT-IDH1, ectopic expression of the IDH1-T157A mutant significantly reduces the glycolytic peak in S phase. Various cell lines generated in FIG. 17H were synchronized by nocodazole blockage for 20 hours and released at the indicated time points, followed by ECAR measurements with Seahorse XF extracellular flux analyzer. n=3, * p<0.05, ** p<0.01. FIG. 17J is a graph showing that, compared to WT-IDH1, ectopic expression of the IDH1-T157A mutant is incapable of reducing TCA cycle in S phase. Various cell lines generated in FIG. 17H were synchronized by nocodazole blockage for 20 hours and released at the indicated time points, followed by OCR measurements with Seahorse XF extracellular flux analyzer. n=3, *p<0.05, ** p<0.01. FIG. 17K depicts immunoblots of the indicated proteins in HeLa stable cell lines. FIG. 17L is a graph showing that, compared to WT-IDH1, ectopic expression of the IDH1-T157A mutant retards cell growth. FIG. 17M depicts colony formation assays showing that, compared to WT-IDH1, ectopic expression of the IDH1-T157A mutant compromises transformation ability. Representative images showing colony growth or anchorage independent growth. FIGS. 17N and 17O are graphs depicting quantification of colony formation results derived from FIG. 17M. ***P<0.001 (mean±s.e.m, n=3).

FIGS. 18A-18L show that Skp2 dedicates the metabolic phenotypes of prostate cancer cell lines in part by promoting IDH1 degradation. FIG. 18A depicts immunoblots showing that there is an inverse correlation between the protein abundance of Skp2 and IDH1 in a panel of prostate cancer (PrCa) cell lines. Immunoblot analysis of C4-2, DU145, LNCaP, PC3, 22-Rv1 and VCaP with the indicated antibodies. FIG. 18B is a graph showing ECAR analysis of different prostate cancer cell lines as listed in FIG. 18A. n=3, * p<0.05, ** p<0.01. FIG. 18C is a graph showing OCR analysis of different prostate cancer cell lines as listed in FIG. 18A. n=3, * p<0.05, ** p<0.01. FIG. 18D depicts immunoblots showing that depletion of endogenous SKP2 in Skp2^{high} cells leads to a significant elevation of IDH1 protein abundance. Two Skp2^{high} cells, PC3 and DU145 were infected with pLKO-shSkp2 or shControl lenti-viruses, selected for 3 days, and harvested for immunoblot analysis. FIG. 18E is a graph showing ECAR analysis of PC3 and DU145 with or without depletion of endogenous SKP2. * p<0.05, ** p<0.01. FIG. 18F is a graph showing OCR analysis of PC3 and DU145 with or without depletion of endogenous SKP2. * p<0.05. FIG. 18G depict immunoblots showing that enforced ectopic expression of Skp2 in Skp2^{low} cells leads to elevated IDH1 degradation. Skp2^{low} cells, LNCaP, C4-2 and 22-Rv1 were infected with HA-Skp2 or GFP lenti-viruses, selected with puromycin for 3 days, and harvested for immunoblot analysis. FIG. 18H is a graph showing ECAR analysis of LNCaP, C4-2 and 22-Rv1 with or without ectopic expression of Skp2. * p<0.05. FIG. 18I is a graph showing OCR analysis of LNCaP, C4-2 and 22-Rv1 with or without ectopic expression of Skp2. * p<0.05, ** p<0.01.

FIG. 18J depicts immunoblots showing that treatment with Skp2 inhibitor SKPin C1 leads to a robust accumulation of IDH1 and IDH2 in cells. 22-Rv1 cells were treated with 0, 1, 3, 10, or 30 μM SKPin C1 for 24 hours, and then harvested for immunoblot analysis. FIG. 18K depicts colony formation assays showing that SKPin C1 blocks the colony growth of protest cancer cells, LNCaP and 22-Rv1. LNCaP and 22-Rv1 cells were plated in 6-well plate (1500 cell/well), treated with 0, 0.1, 0.3, or 1 μM Skin C1 for 7 days, and changed to fresh media for another 3 weeks. FIG. 18L is a graph depicting depletion of endogenous IDH1 or IDH2 abolishes the effects of SKPin C1 on OCR. HAP1-IDH1^{-/-}, HAP1-IDH2^{-/-} and parental cells were treated with 3 μM SKPin C1 for 24 hours, followed by OCR analysis by Seahorse XF 24 analyzer. * p<0.05.

### DETAILED DESCRIPTION OF THE INVENTION

The present disclosure features compositions and methods of treating a cancer in a subject by administering to the subject a Skp2 inhibitor and an inhibitor of glycolytic metabolism (e.g., PKM2 inhibitor).

The invention is based, at least in part, on the discovery of a cell cycle-dependent metabolic cycle in mammalian cells through SCF^{Skp2}-mediated IDH1 degradation. Specifically, mammalian cells predominantly utilized the TCA cycle in G1 phase, but preferred glycolysis in S phase. Mechanistically, coupling cell cycle with metabolism was largely achieved by timely destruction of IDH1/2, which are key TCA cycle enzymes, in a Skp2-dependent manner. As such, depleting SKP2 abolished cell cycle-dependent fluctuation of IDH1/2 expression, leading to reduced glycolysis in S phase. Thus, SCF^{Skp2} controls IDH1/2 stability to ensure timely shift from TCA cycle to glycolysis during G1 to S cell cycle transition.

Whether glucose is predominantly metabolized via oxidative-phosphorylation or glycolysis differs between quiescent versus proliferating cells, including tumor cells. Given the high demand of biomacromolecules, including lipid, nucleotides and amino acids, to prepare for DNA replication and subsequent cell division, high rates of glycolysis and low rates of TCA cycle enable more flux of intermediates into the biomass synthesis pathways. Indeed, several lines of evidence advocate a bi-directional interplay between the cell cycle and metabolic machineries. On one hand, key metabolic enzymes are directly regulated in a cell cycle-dependent manner, such as PFKFB3 (6-phosphofructo-2-kinase/fructose-2, 6-bisphosphatase-3) by SCF^{GRR1,} SCF^{β-TRIP} and APC^{Cdh1}, HK2 (hexokinase 2) by cyclin D1, PFKP and PKM2 by CDK6/cyclin D3, and GLS1 by APC^{Cdh1}. On the other hand, disturbing metabolism also could compromise cell cycle progress.

The present study therefore reveals a novel oncogenic role of Skp2 independent of its other biological substrate p27 in cell cycle regulation, by promoting the metabolic switch from utilization of TCA cycle to glycolysis. In one example, elevated Skp2 abundance in prostate cancer cells destabilized IDH1 to favor glycolysis and subsequent tumorigenesis. Based on these results, targeting Skp2 has the potential to provide a novel anti-cancer therapy in part by suppressing cancer metabolism.

### Therapeutic Combinations of the Invention

The invention provides compositions comprising a therapeutic combination comprising a Skp2 inhibitor and an inhibitor of glycolytic metabolism (e.g., PKM2 inhibitor) and methods of using such compositions for the treatment of cancer (e.g., prostate cancer, breast cancer and glioblastoma).

### Therapeutic Methods

The methods and compositions provided herein can be used to treat or prevent progression of a cancer (e.g., breast cancer, prostate cancer, glioblastoma) using a Skp2 inhibitor and an inhibitor of glycolytic metabolism (e.g., PKM2 inhibitor).

Compositions of the invention are administered to subjects, particularly humans, suffering from, having, susceptible to, or at risk of developing cancer (e.g., breast cancer, prostate cancer, glioblastoma). Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g. opinion) or objective (e.g., measurable by a test or diagnostic method).

In one embodiment, a therapeutic combination of the invention comprises an effective amount of a Skp2 inhibitor and an effective amount of a PKM2 inhibitor. If desired, such therapeutic combinations are administered in combination with standard chemotherapeutics. Methods for administering combination therapies (e.g., concurrently or otherwise) are known to the skilled artisan and are described for example in Remington's Pharmaceutical Sciences by E. W. Martin.

### Pharmaceutical Compositions

Pharmaceutical compositions of the invention contain a Skp2 inhibitor and an inhibitor of glycolytic metabolism (e.g., PKM2 inhibitor). Typically, such compositions comprise an effective amount of an agent that inhibits the expression or activity in a physiologically acceptable carrier. Therapeutic combinations of the invention are typically formulated and administered separately, but may also be combined and administered in a single formulation.

Typically, the carrier or excipient for the composition provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like.

The administration may be by any suitable means that results in a concentration of the therapeutic that, combined with other components, is effective in ameliorating, reducing, or stabilizing the disease symptoms in a subject. The composition may be administered systemically, for example, formulated in a pharmaceutically-acceptable buffer such as physiological saline. Preferable routes of administration include, for example, subcutaneous, intravenous, intraperitoneally, intramuscular, intrathecal, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of cancer, although in certain instances lower amounts will be needed because of the increased specificity of the agent. A composition is administered at a dosage that ameliorates or decreases effects of the cancer as determined by a method known to one skilled in the art.

The therapeutic or prophylactic composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intrathecally, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent to a particular cell type. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic agent is formulated with appropriate excipients into a pharmaceutical composition that, upon administration, releases the therapeutic agent in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac dysfunction or disease, the composition may include suitable parenterally acceptable carriers and/or excipients. The active therapeutic agent(s), including a a Skp2 inhibitor and an inhibitor of glycolytic metabolism (e.g., PKM2 inhibitor) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic agent is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Inhibitory Nucleic Acids

Inhibitory nucleic acid molecules that inhibit the expression or activity of Skp2 or PKM2 are useful for the treatment of cancer in the methods of the invention. Such oligonucleotides include single and double stranded nucleic acid molecules (e.g., DNA, RNA, and analogs thereof) that bind a nucleic acid molecule that encodes a Skp2 or PKM2 polypeptide (e.g., antisense molecules, siRNA, shRNA), as well as nucleic acid molecules that bind directly to the polypeptide to modulate its biological activity (e.g., aptamers). Inhibitory nucleic acid molecules described herein are useful for the treatment of cancer (e.g., breast cancer, glioblastoma, prostate cancer).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer.

The inhibitory nucleic acid molecules of the present invention may be employed as double-stranded RNAs for RNA interference (RNAi)-mediated knock-down of expression. In one embodiment, expression of Skp2 polypeptide and/or PKM2 polypeptide is reduced in a subject having cancer. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances, the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo (e.g., a nucleic acid encoding Skp2 or PKM2). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Alternatively, expression of Skp2, PKM2, or both, may be inhibited, or silenced by introducing vectors encoding Clustered regularly interspaced short palindromic repeats (CRISPR)/Cas9 nuclease engineered to target Skp2, PKM2, or both.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus, the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Polynucleotides

Naked polynucleotides, or analogs thereof, are capable of entering mammalian cells and inhibiting expression of a gene of interest (e.g., a SKP2 or PKM2 polynucleotide). Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Diagnostics

The present invention features assays for the detection of Skp2, IDH1, and/or IDH2 protein levels or activity. In other embodiments, the invention features assays for characterizing metabolism (e.g., glycolysis, TCA activity). Levels Skp2, IDH1, and/or IDH2 are measured in a subject sample (e.g., tumor biopsy) and used to select patient therapies (e.g., treatment with Skp2 and/or PKM2 inhibitors). Standard methods may be used to measure levels of Skp2, IDH1, and/or IDH2 in any biological sample. Such methods include immunoassay, ELISA, western blotting and radioimmunoassay.

The diagnostic methods described herein can be used individually or in combination with any other diagnostic method known in the art.

### Kits

The invention provides kits for the treatment or prevention of cancer. In some embodiments, the kit includes a therapeutic composition containing a Skp2 inhibitor and an inhibitor of glycolytic metabolism (e.g., PKM2 inhibitor) in unit dosage form. In other embodiments, the Skp2 inhibitor and inhibitor of glycolytic metabolism (e.g., PKM2 inhibitor) are provided in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: SCF^{Skp2} Dictates Cell Cycle-Dependent Metabolic Oscillation Between Glycolysis and TCA Cycle

Actively proliferating cancer cells are addicted to glycolysis despite the presence of oxygen, whereas normal differentiated cells largely rely on oxidative phosphorylation (OXPHOS) (Warburg, Science 123, 309 (1956)). For cancer cells, this phenotype is termed the “Warburg Effect”, which has been shown to benefit cancer cell growth and tumorigenesis (Warburg, Science 123, 309 (1956)). Clinically, increased glycolysis in cancer cells is accompanied by robust glucose uptake, which underlies the usage of fluorodeoxyglucose positron emission tomography (FDG-PET) for tumor diagnosis and response to cancer therapy (Boellaard et al., European journal of nuclear medicine and molecular imaging 42, 328 (2015)). Mechanistic investigations reveal that unlike non-proliferating cells, actively dividing cells, including tumor cells, incorporate intermediates of glycolysis into the macromolecules (e.g. non-essential amino acids, fatty acids and nucleotides) to facilitate cell growth and division, a process tightly controlled by many oncogenic signaling pathways, involving PKM2, HIF, Akt, Ras and Myc as important regulatory components (Vander Heiden et al., Science 324, 1029 (2009); Christofk et al., Nature 452, 230 (2008); Manning et al., Cell 129, 1261 (2007); Gordan et al., Cancer cell 12, 108 (2007); Dang et al., Trends in biochemical sciences 24, 68 (1999); Bensaad et al., Cell 126, 107 (2006); Kaelin et al., Molecular cell 30, 393 (2008); Shi et al., Mot Cancer 8, 32 (Jun. 5, 2009)). However, the molecular underpinnings responsible for the distinct metabolic dependence between proliferating and non-proliferating cells remain largely unknown.

Intriguingly, a metabolic cycle has been reported in yeasts that is coupled with cell cycle events (Tu et al., Science 310, 1152 (2005)). A shift from the tricarboxylic acid (TCA) cycle to glycolysis in S phase in yeast was found to minimize intracellular reactive oxygen species (ROS) production, possibly to avoid damage to newly duplicated DNA (Chen et al., Science 316, 1916 (2007)). Although a previous study indicates crosstalk between cell cycle regulators and glycolysis (Tudzarova et al., P Natl Acad Sci USA 108, 5278 (Mar. 29, 2011)), the exact molecular mechanism that governs a similar coupling of metabolism to the cell-cycle in mammalian cells remains elusive.

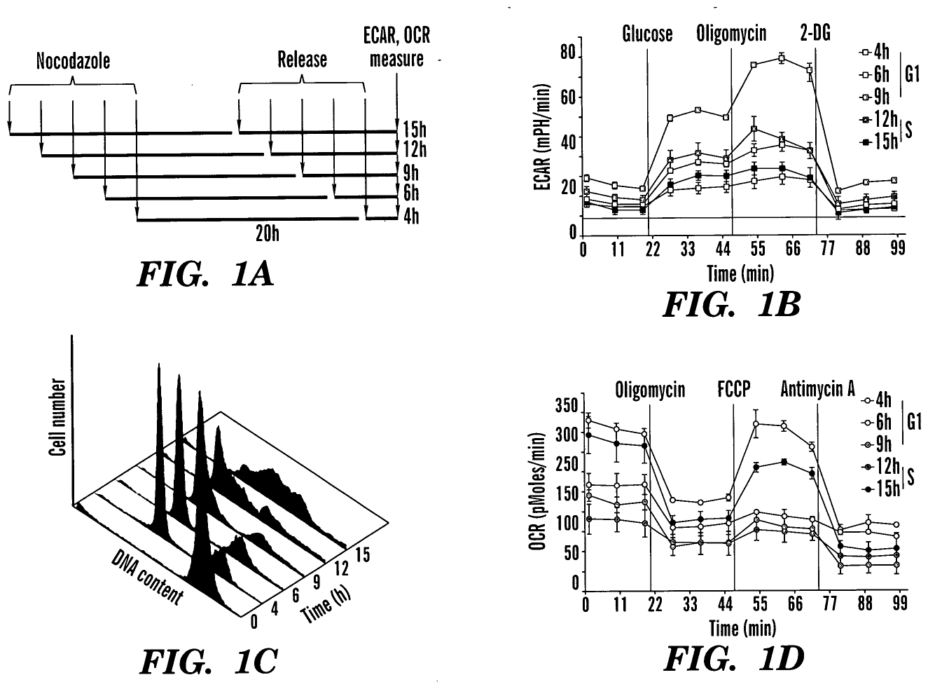

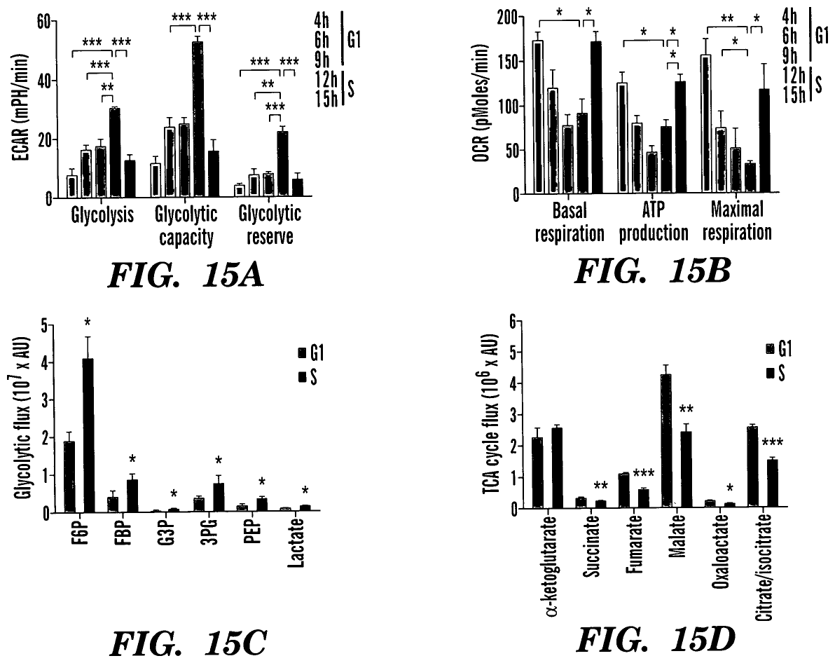

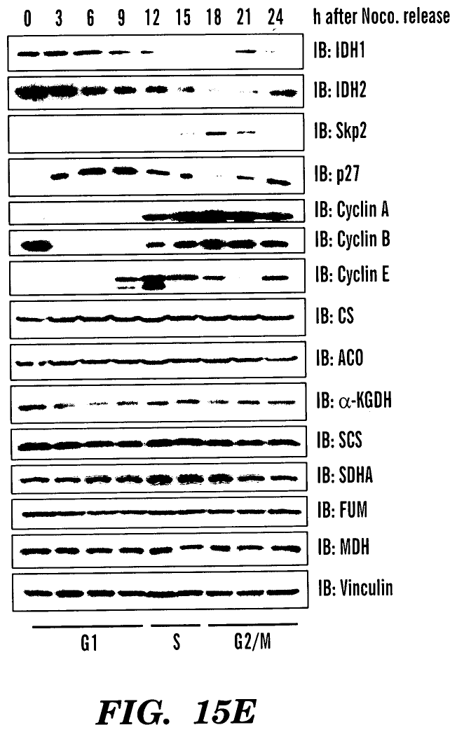

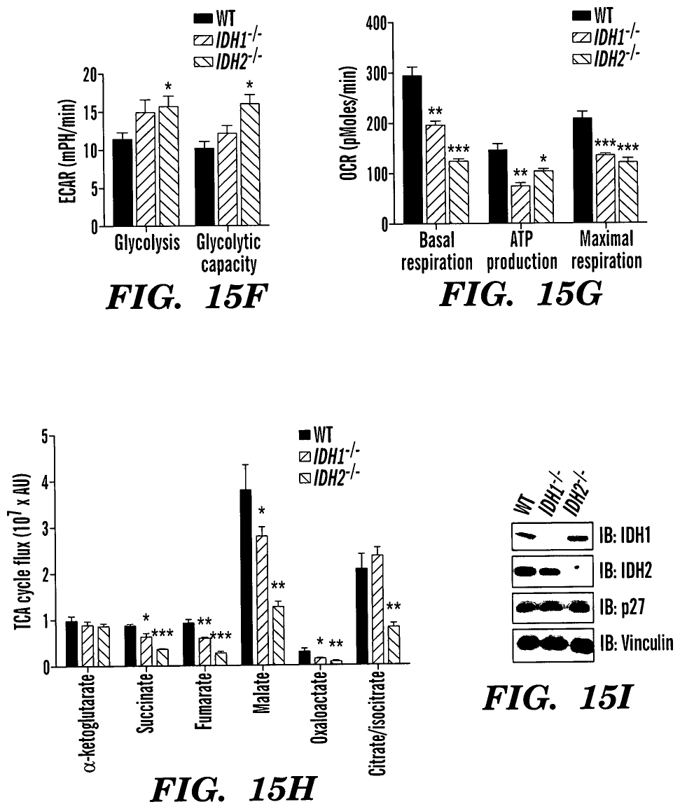

To understand the molecular mechanisms that govern coupling of metabolism to the cell-cycle in mammalian cells, rates of glycolysis (indicated by extracellular acidification rate, ECAR) and TCA cycle activity (indicated by oxygen consumption rate, OCR) were measured at different cell cycle phases (FIG. 1A). It was observed that glycolysis peaked in early S phase (FIGS. 15A and 1B-1C), accompanied with a relatively lower rate of TCA cycle (FIGS. 15B and 1D). Without being bound by theory, these findings indicate that glucose metabolism is regulated in a cell-cycle dependent manner in mammalian cells. This may be in part due to metabolic needs, where cells rely mostly on the TCA cycle during G1 phase, while switching to glycolysis, a less economic form, to accumulate intermediate metabolites that used as building blocks for macromolecules synthesis to accumulate biomass for subsequence DNA replication and cytokinesis (Pavlova et al., Cell metabolism 23, 27 (2016)).

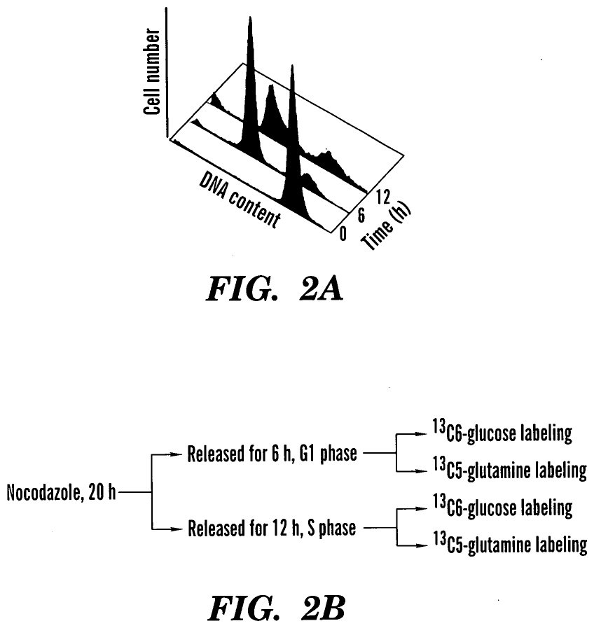

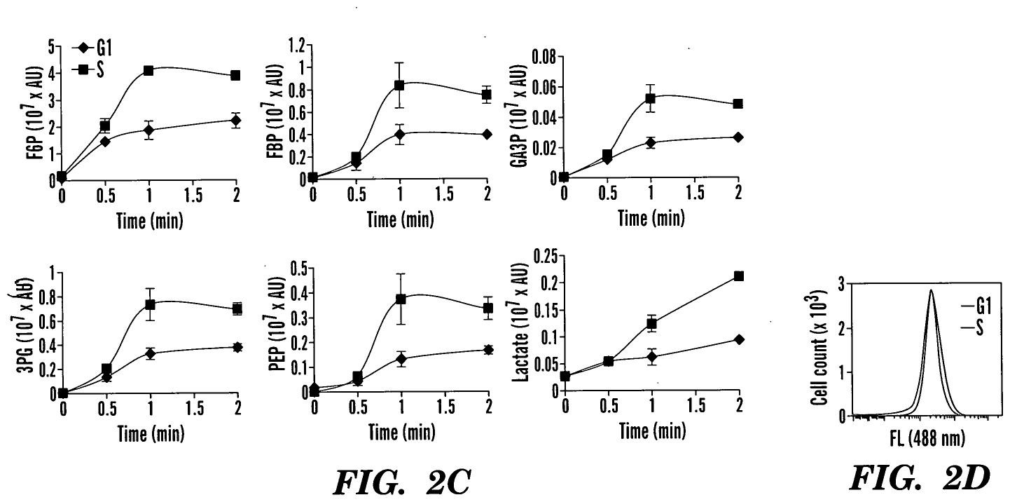

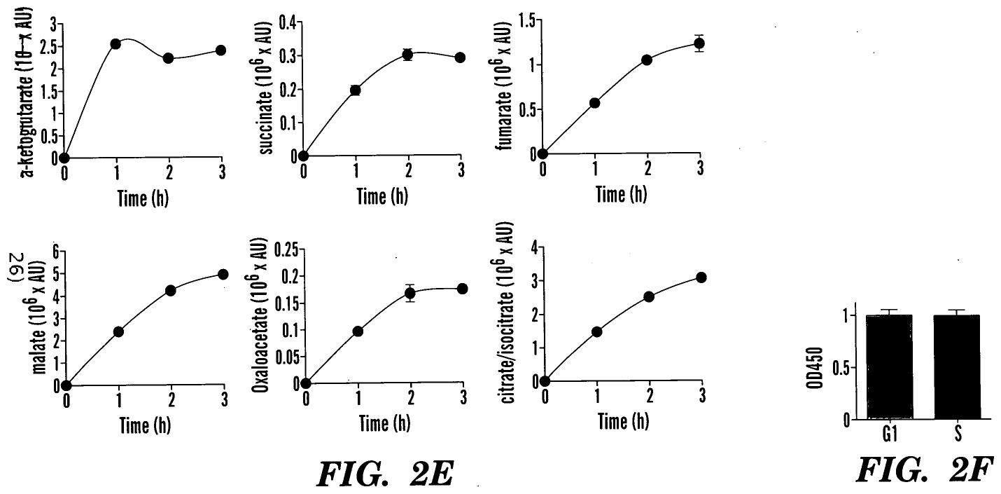

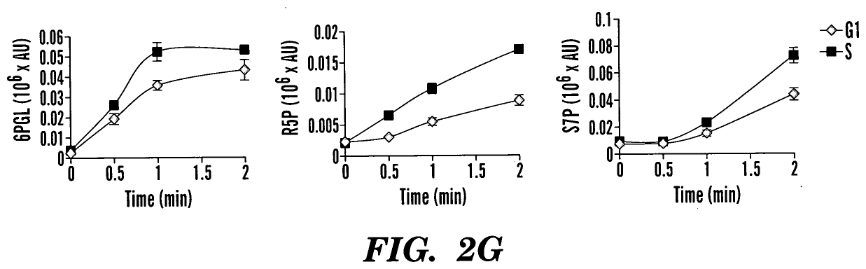

To further explore this cell cycle-dependent metabolic shift between glycolysis and TCA cycle, cells were synchronized and released into either G1 or S phase (FIG. 2A), followed by labeling with ¹³C6-glucose or ¹³C5-glutamine for profiling metabolic intermediates with LC-MS (FIG. 2B) (Yuan et al., Nature protocols 7, 872 (2012)). Notably, cells in S phase exhibited a higher glycolytic flux rate than cells in G1 phase (FIGS. 15C and 2C), which was not explained by differences of glucose uptake in these two cell cycle phases (FIG. 2D). In contrast to the relatively fast glycolysis flux, the TCA cycle flux took approximately two hours to reach a steady-state (FIG. 2E). In keeping with the metabolic switch from TCA cycle to glycolysis in S phase, a reduction of TCA cycle flux was observed for cells in S phase compared to cells in G1 phase (FIG. 15D), which appeared to be independent of glutamine uptake changes (FIG. 2F). Furthermore, flux through the pentose phosphate pathway (PPP) revealed by ¹³C6-glucose labeling was also relatively higher for cells in S phase than in G1 phase, consistent with elevated synthesis of fatty acid, aromatic amino acids, and nucleic acid, which is coupled with DNA duplication events in S phase (FIG. 2G). Without being bound by theory, these results support a model for cell cycle-dependent metabolic switch from TCA cycle to glycolysis in S phase to facilitate DNA duplication and cell growth.

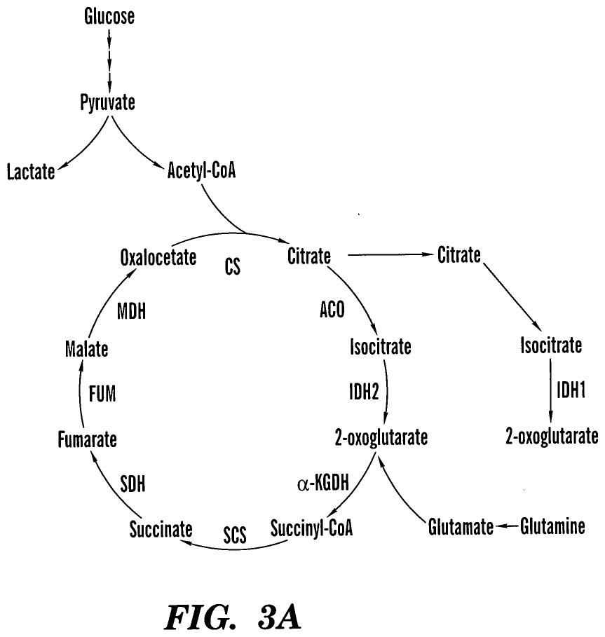

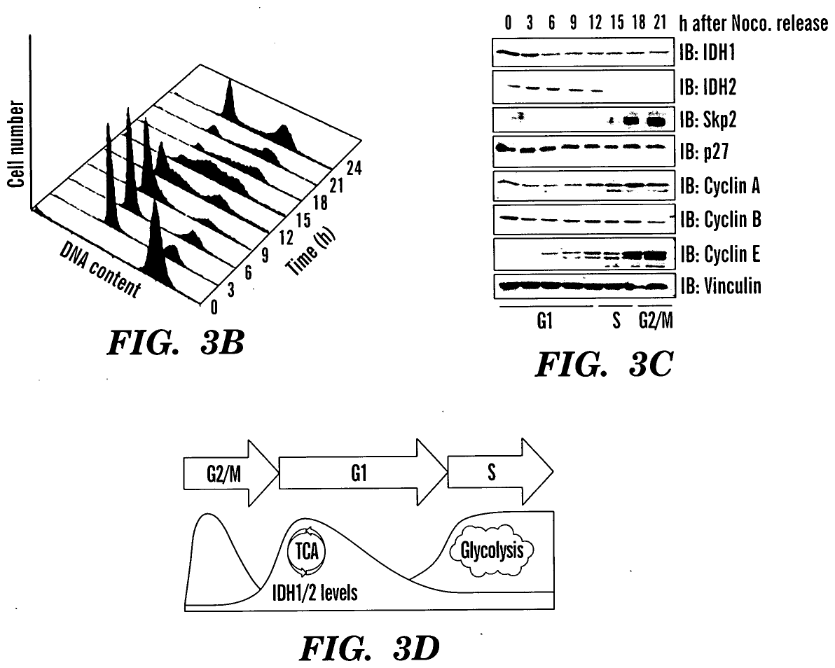

The rate of TCA cycle is primarily governed by a cohort of essential enzymes (FIG. 3A) (Srere, Annual review of biochemistry 56, 89 (1987)). Among them, it was found that the protein abundance of IDH1 and IDH2, but not other TCA cycle enzymes, fluctuated during the cell cycle, with both being relatively lower in S phase (FIGS. 15E and 3B-3D). Three IDH isoenzymes exist in mammalian cells, among which NADP⁺-dependent IDH2 and NAD⁺-dependent IDH3 are located within mitochondria to catalyze the conversion of isocitrate to α-ketoglutarate, while the cytosolic NADP⁺-dependent IDH1 catalyzes the same reaction using cytosolic citrate (Kim et al., Biochimica et Biophysica Acta (BBA)-Molecular Basis of Disease 1842, 135 (2014); Itsumi et al., Cell Death & Differentiation 22, 1837 (2015)). Notably, both Idh1 and Idh2 knockout mice are viable and fertile, with noticeable mitochondrial dysfunction and increased oxidative stress, indicating that IDH1 and IDH2 might partially compensate for each other's function in vivo (Kim et al., Biochimica et Biophysica Acta (BBA)-Molecular Basis of Disease 1842, 135 (2014); Itsumi et al., Cell Death & Differentiation 22, 1837 (2015)).

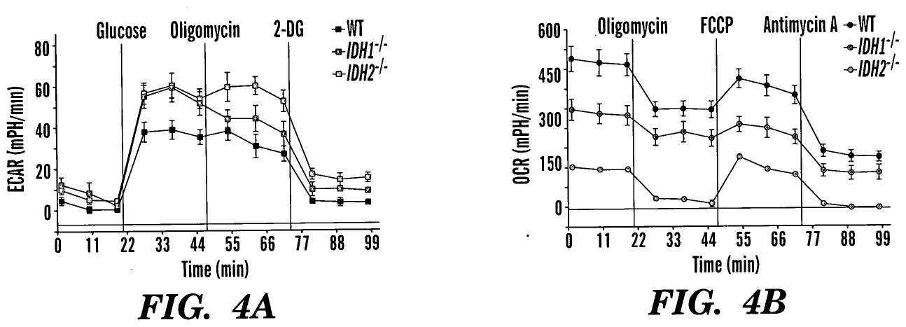

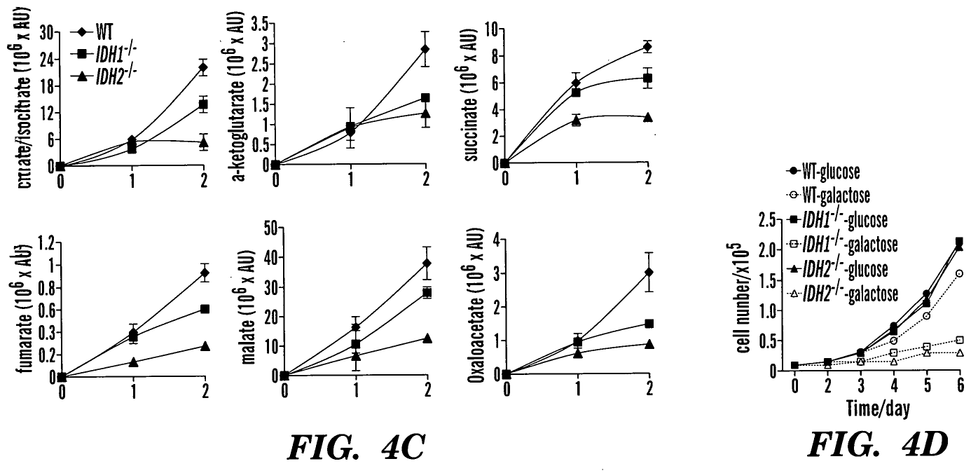

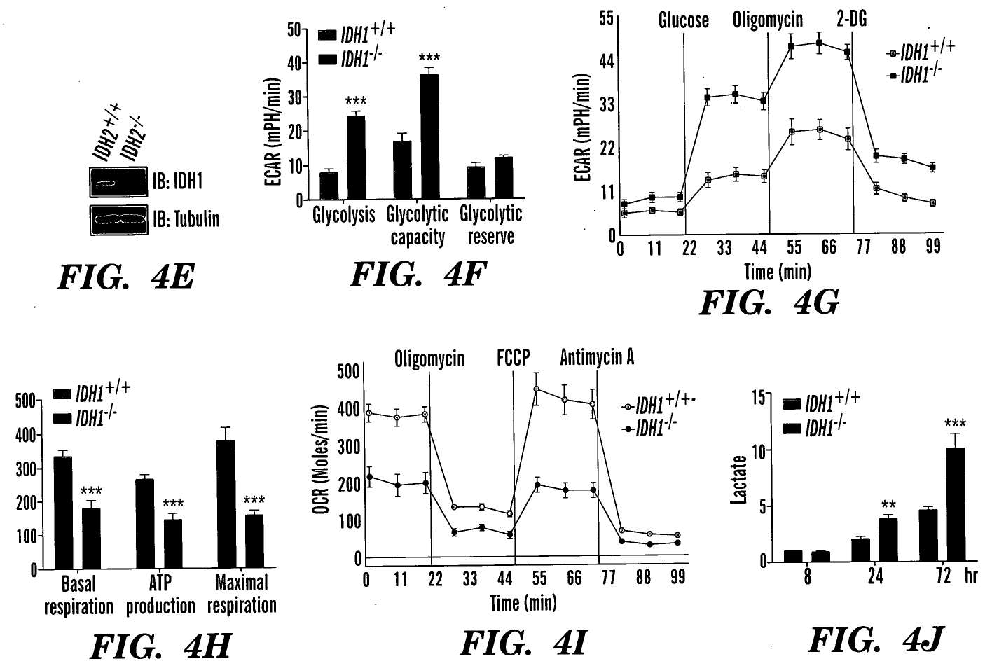

To understand the importance of fluctuations of IDH1 and IDH2 during the cell cycle, IDH1 and IDH2 knockout haploid HAP1 cells were generated by CRISPR/Cas-9. IDH2 knockout cells, and with a similar trend, the IDH1 knockout cells, displayed increased glycolysis and compromised mitochondrial respiration (FIGS. 15F-15G and 4A-4B). Similarly, compared to wild-type cells, the TCA cycle flux in IDH2⁻, and to a lesser extent, IDH1^{-/-} cells were compromised (FIGS. 15H-15I and 4C). Oxidative phosphorylation deficient cells are viable in glucose-rich media, but not in galactose-rich media, termed as “metabolic state-dependent lethality” (Gohil et al., Nature biotechnology 28, 249 (2010)). As such, similar to loss of mitochondrial IDH2, loss of the cytosolic IDH1, led to growth arrest in galactose-rich media (FIG. 4D). Notably, compared to wild-type cells, CRISPR-mediated depletion of endogenous IDH1 in HeLa cells also led to increased glycolysis (FIGS. 4E-4G), reduced TCA cycle metabolism (FIGS. 4H-4I), and increased lactate production (FIG. 4J). Without being bound by theory, these data indicate that cytosolic IDH1, together with mitochondrial IDH2, play essential roles in governing TCA cycle metabolism.

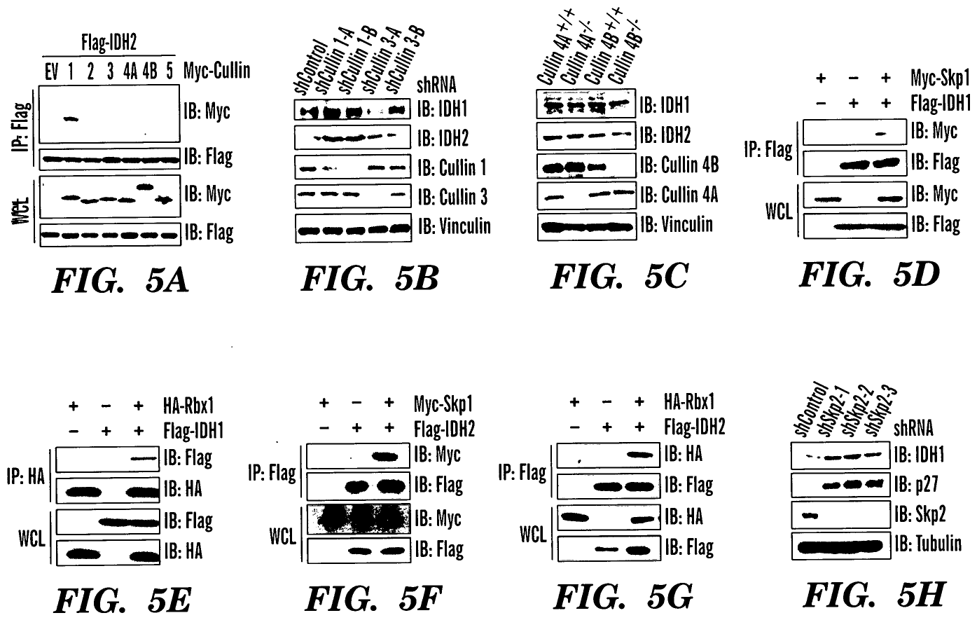

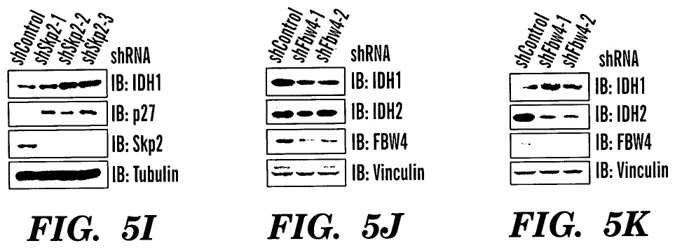

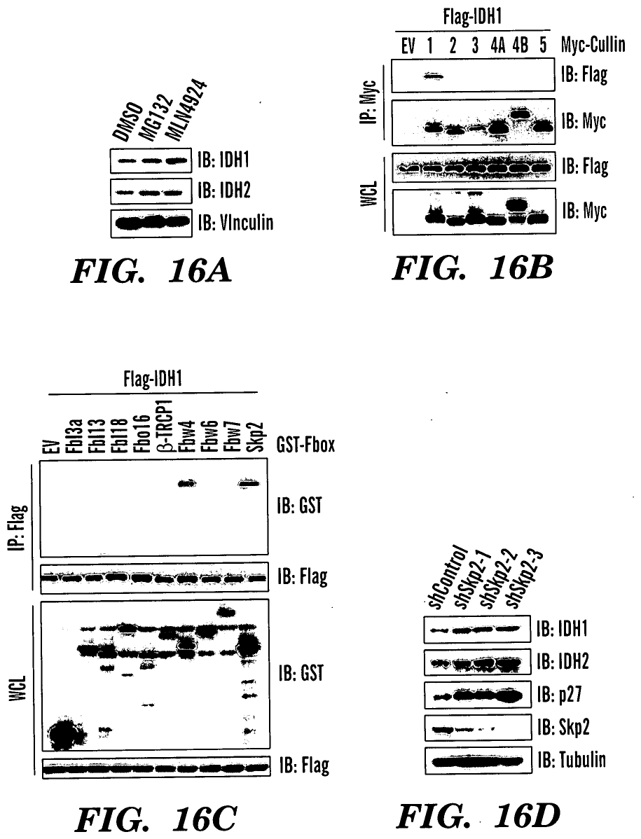

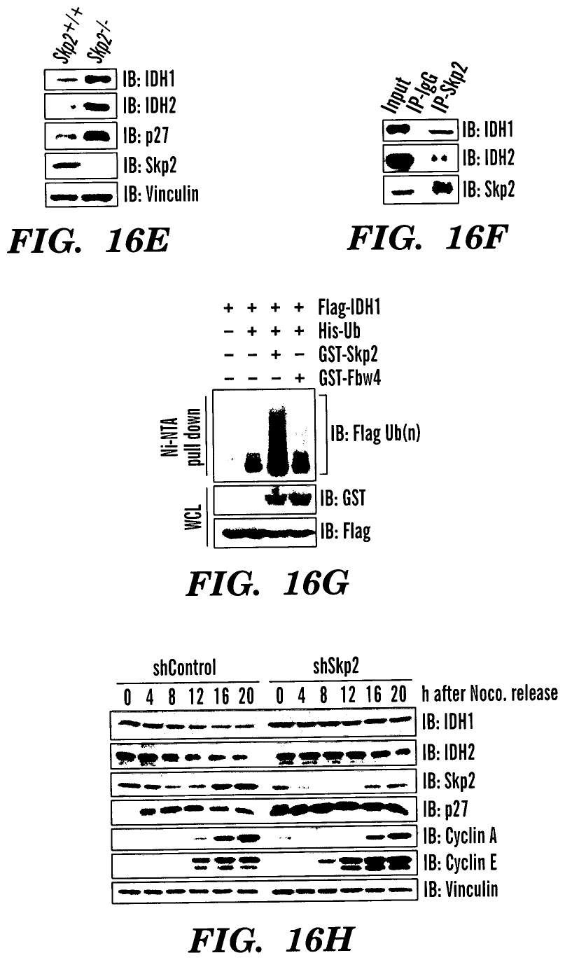

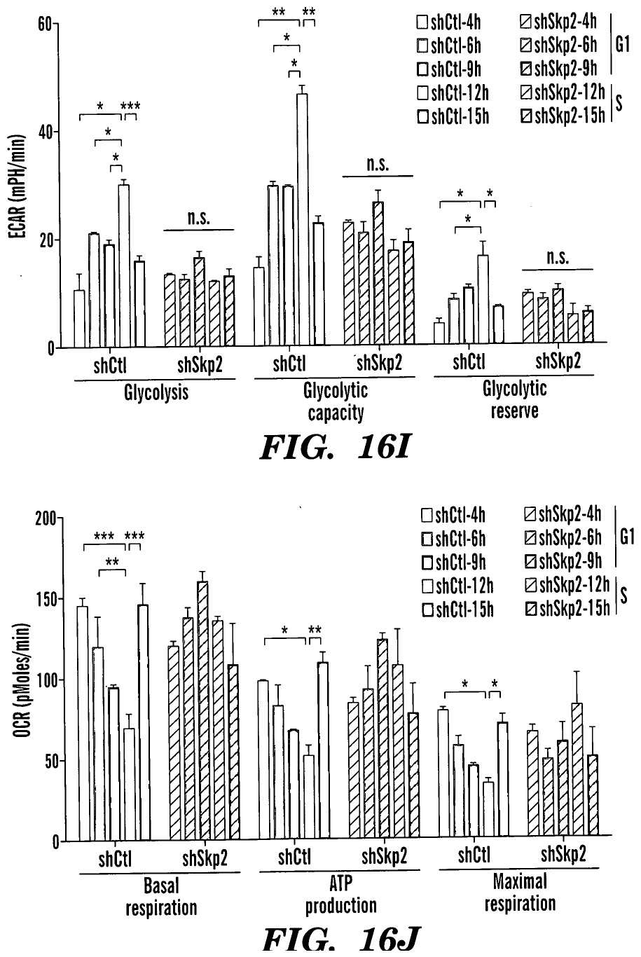

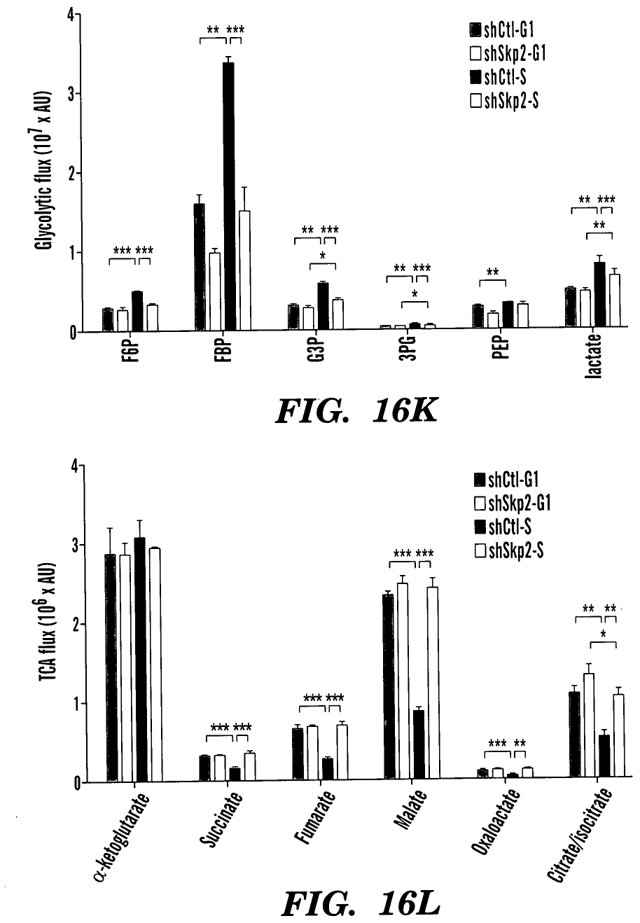

To identify the E3 ubiquitin ligase(s) responsible for S-phase specific degradation of IDH1/2, IDH1 and IDH2 protein levels were determined. Endogenous IDH1 and IDH2 protein abundance were elevated in cells treated with either the proteosome inhibitor MG132 or the Cullin neddylation inhibitor, MLN4924 (FIG. 16A). Without being bound by theory, this at least implicates a Cullin-based E3 ligase in the control of IDH1/2 degradation. Furthermore, IDH1 and IDH2 preferentially interacted with Cullin 1 among various Cullins examined (FIGS. 16B and 5A). Moreover, depleting Cullin 1, but not Cullin 3, Cullin 4A, nor Cullin 4B, led to IDH1 and IDH2 accumulation (FIGS. 5B-5C). In further support of a Cullin 1-containing E3 ligase(s) regulating IDH1 and IDH2 stability, two other essential components of the canonical Skp1-Cullinl-F-box (SCF) ubiquitin ligase complex, Skp1 and Rbx1, also interacted with IDH1 and IDH2 (FIGS. 5D-5G). Notably, Flag-tagged IDH1 coimmunoprecipitated with GST-tagged Fbw4 and Skp2 in cells, but not other F-box proteins examined, under ectopic overexpression conditions (FIG. 16C). However, depleting SKP2 with multiple independent shRNAs, but not FBW4, induced IDH1 in multiple cell lines (FIGS. 16D and 5H-5K). More importantly, IDH1 and IDH2 were elevated in Skp2^{-/-} mouse embryonic fibroblasts (MEFs) compared to their wild-type counterparts, further implicating Skp2 as a physiological negative regulator of IDH protein stability (FIG. 16E).

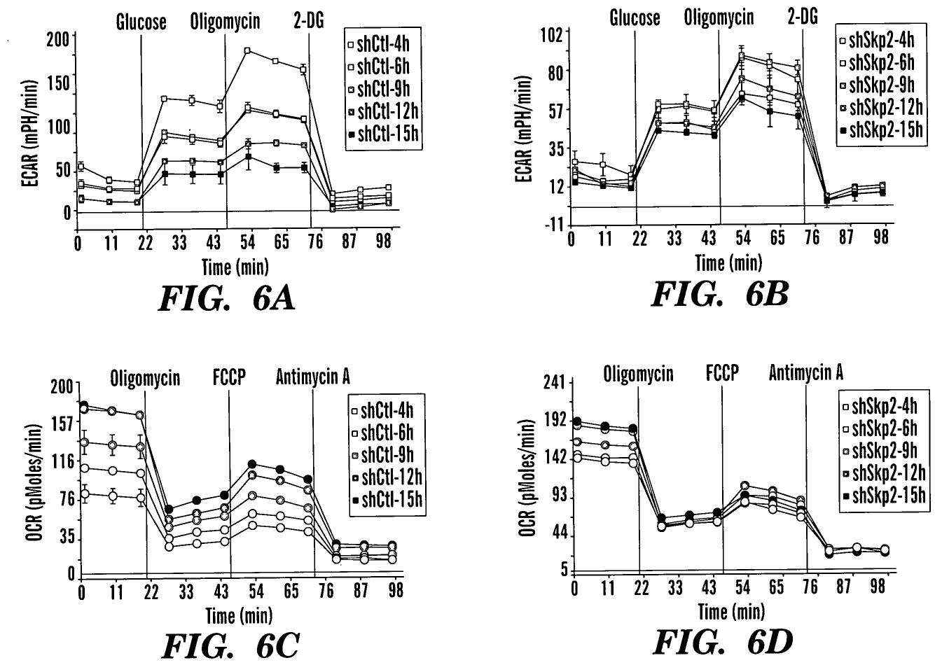

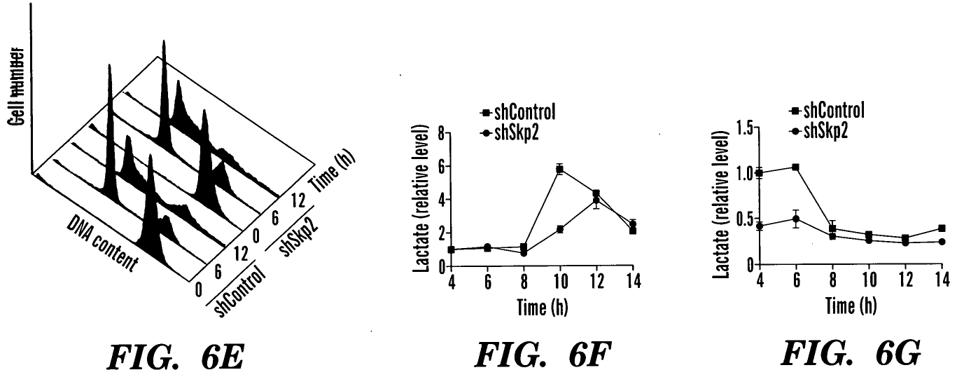

Consistent with this notion, it was found that Skp2, but not Fbw4 was capable of promoting IDH1 ubiquitination in cells (FIG. 16G). Moreover, in support a physiological role of Skp2 in regulating IDH1 protein stability, Skp2 interaction with IDH1 was detected at endogenous levels (FIG. 16F). More importantly, depletion of SKP2 abolished the cell cycle-dependent fluctuation of IDH1/2 protein abundance (FIG. 16H), which correlated with reduced glycolysis (FIGS. 161 and 6A-6B) and OCR oscillation (FIGS. 16J and 6C-6D) in S phase. To exclude the possibility that these metabolic changes were an indirect consequence of a change in cell-cycle distribution due to depletion of SKP2, cells were first synchronized in G1 or S cell cycle phases before performing metabolic studies. It was found that depleting SKP2 also abolished cell cycle-dependent flux changes in glycolytic and TCA cycle intermediates (FIGS. 16K-16L and 6E). Moreover, SKP2 depletion also resulted in a sharp decrease in extracellular lactate levels during S phase, providing further support for a pivotal role of Skp2 in governing the cell cycle-dependent switch to glycolytic metabolism when cells enter S phase (FIG. 6F-6G).

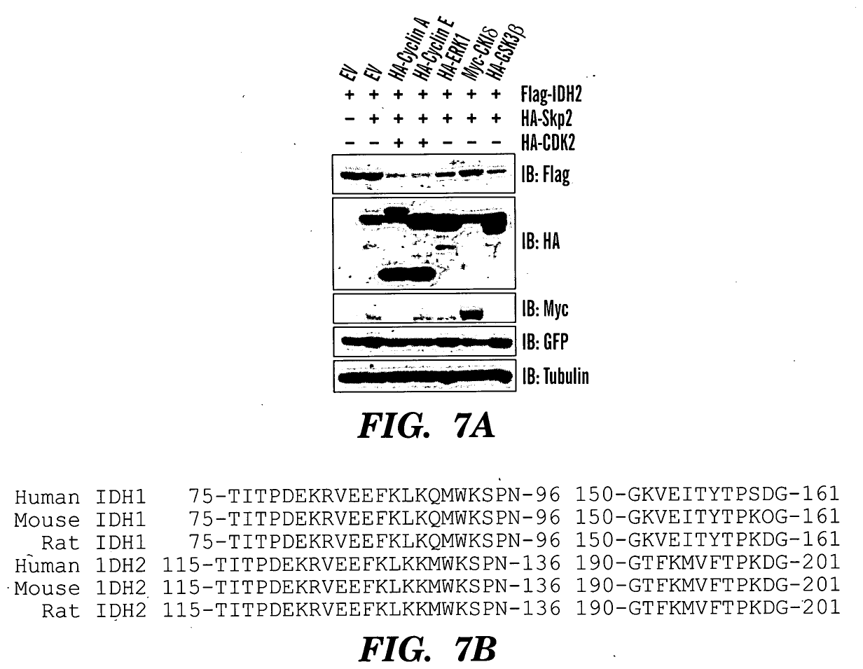

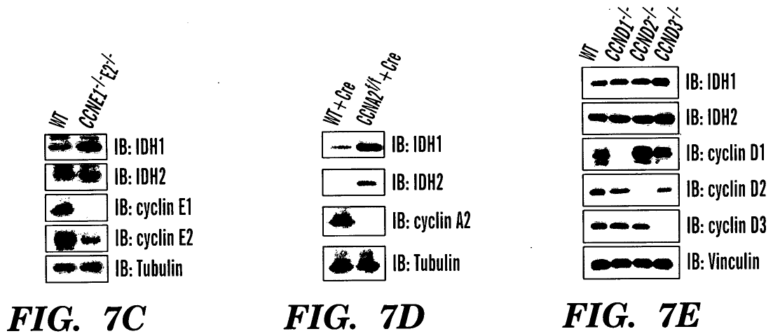

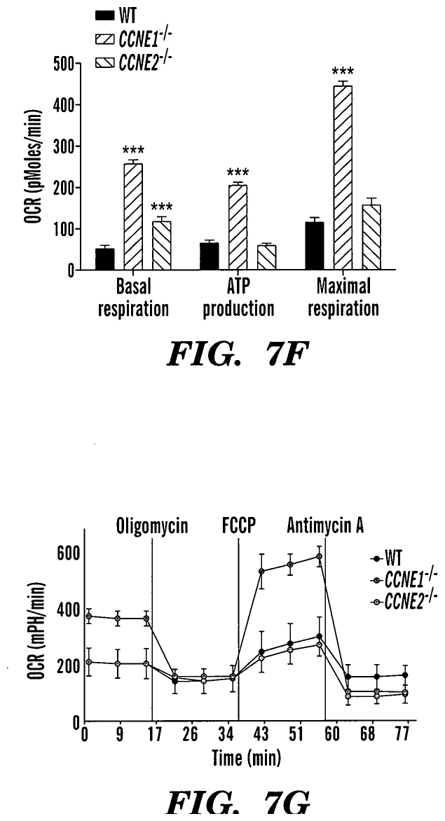

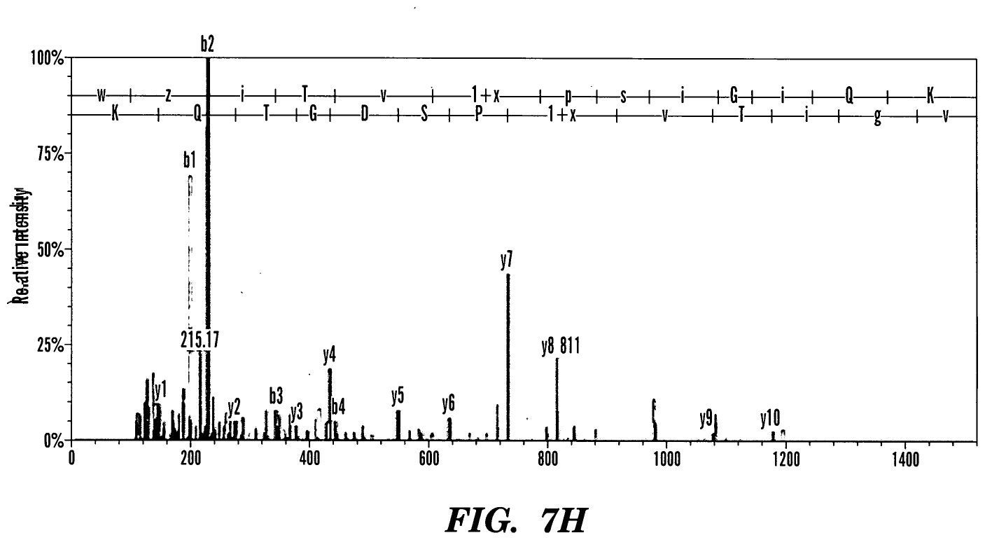

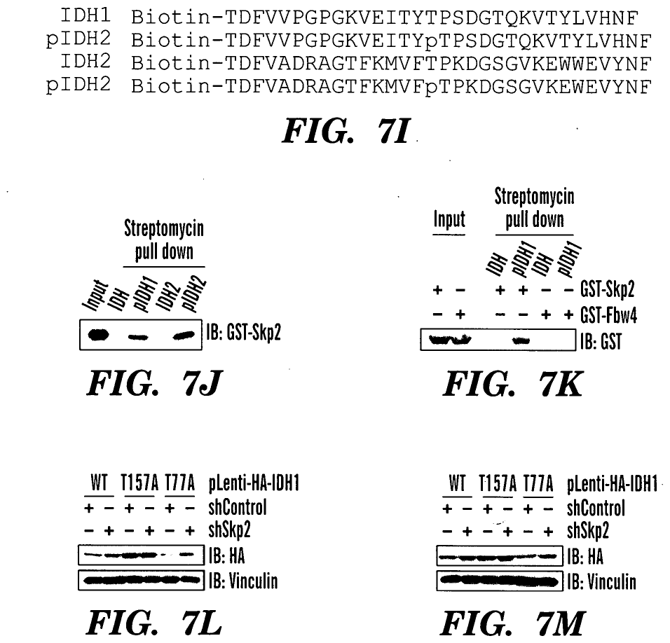

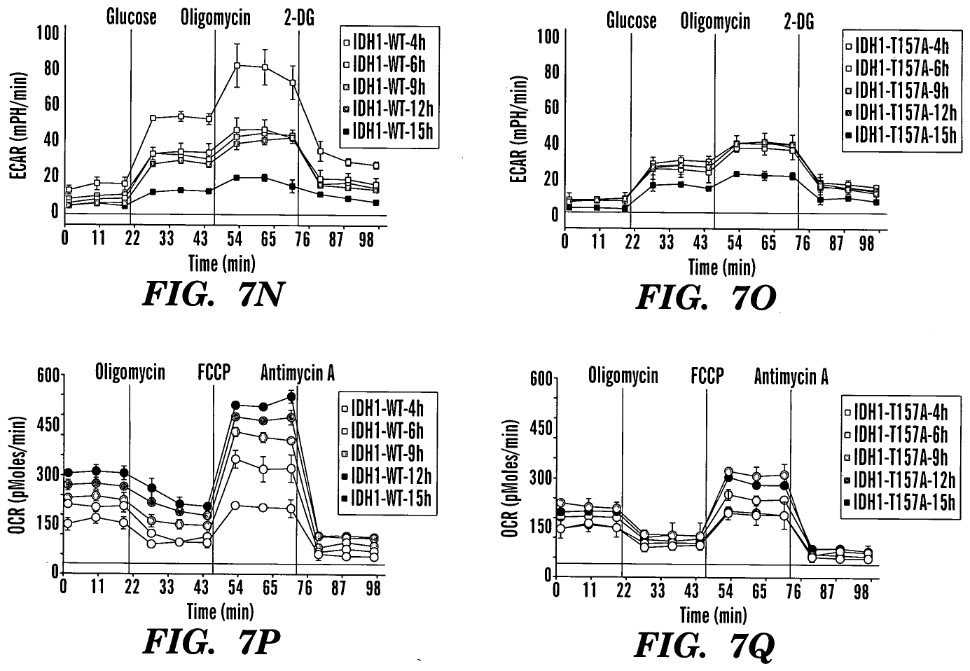

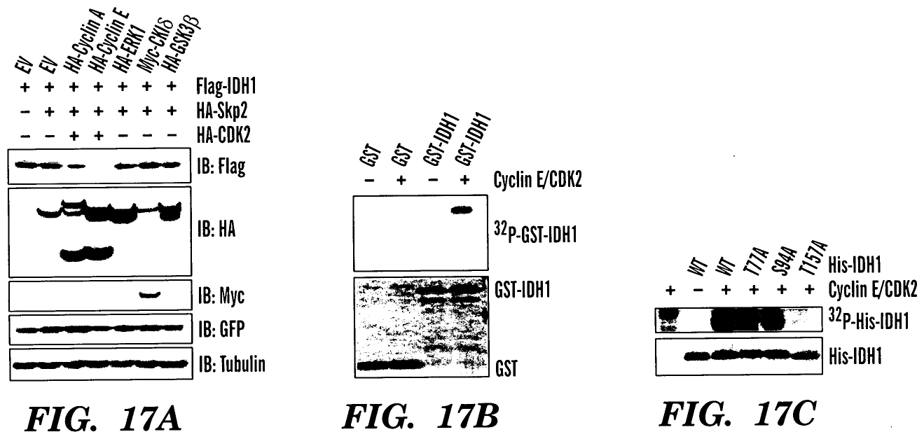

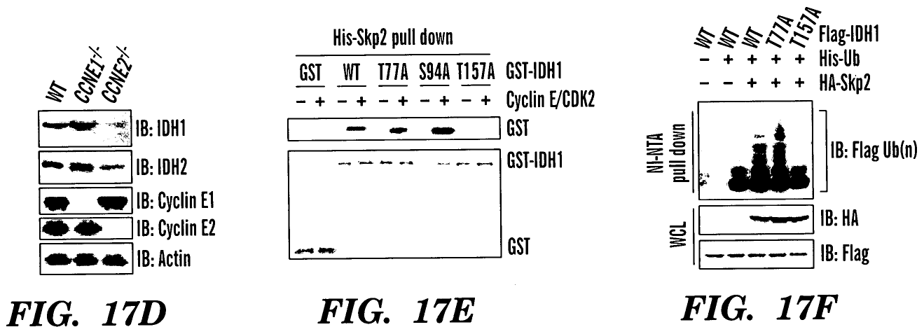

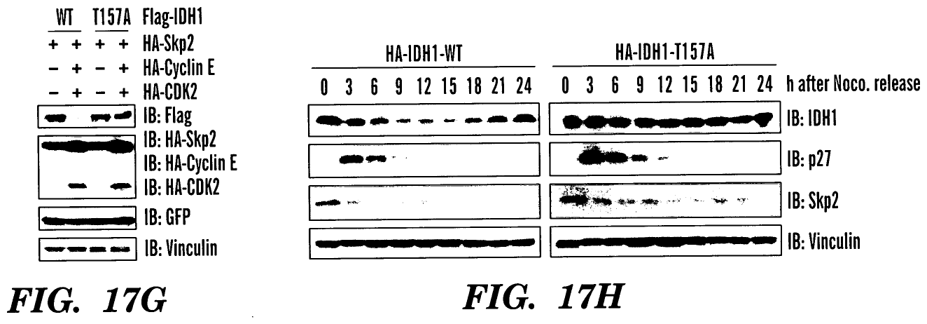

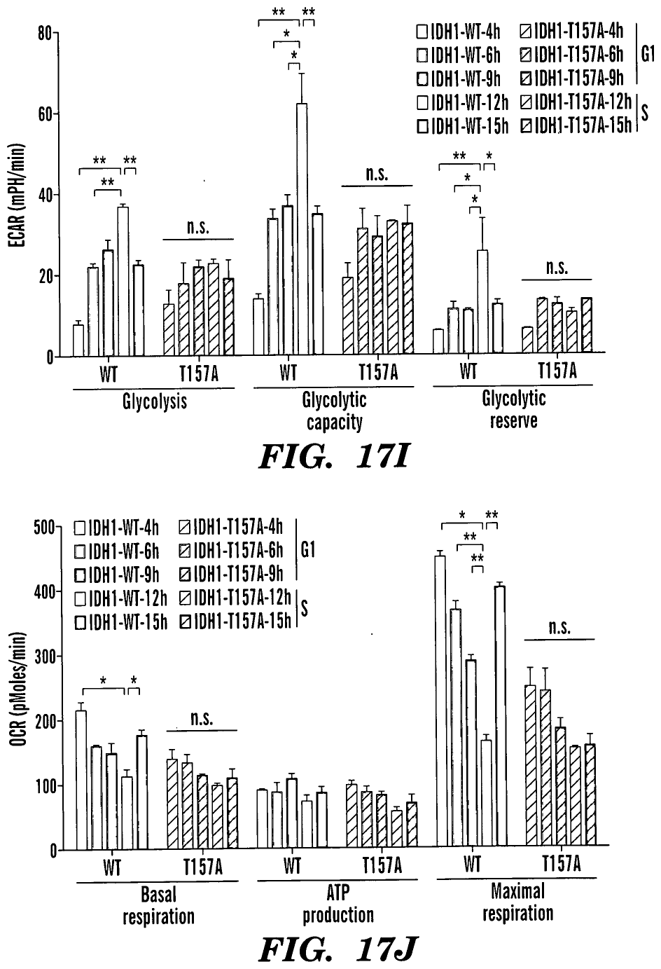

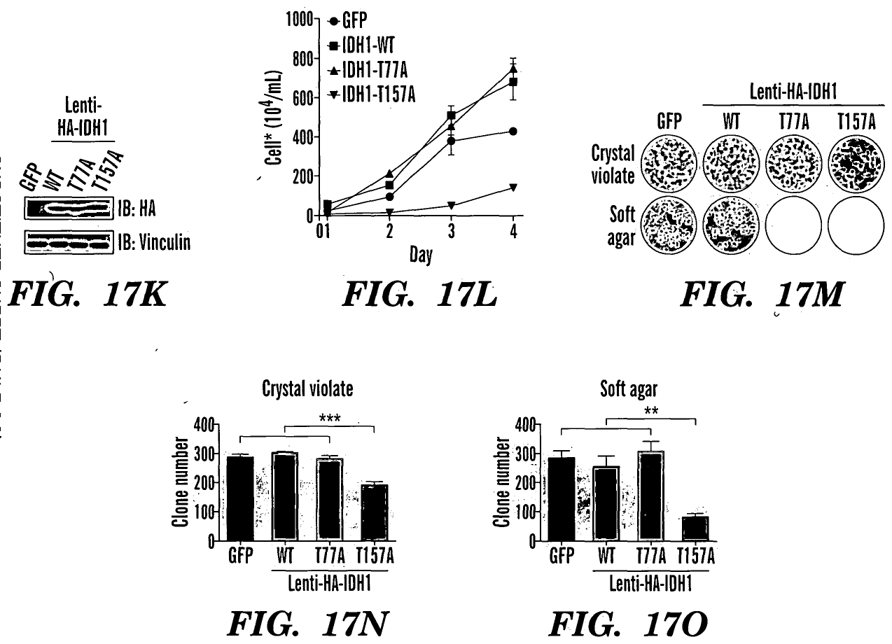

SCF^{Skp2} typically binds and ubiquitinates its downstream substrates in a phosphorylation-dependent manner (Wang et al., Nature reviews Cancer 14, 233 (2014)). Therefore, a panel of modifying kinase(s) potentially involved in Skp2-mediated degradation of IDH1/2 in cells was examined. Notably, cyclin E/CDK2, and to a lesser extent, cyclin A/CDK2, promoted Skp2-mediated degradation of IDH1 and IDH2 in cells (FIGS. 17A and 7A). Further studies revealed that cyclin E/CDK2 phosphorylated IDH1 in vitro primarily at the evolutionarily conserved T157 site that fits in the canonical CDK2 phosphorylation consensus motif (Liu et al., Nature 508, 541 (2014)) (FIGS. 17B-17C and 7B). In support of a physiological role for cyclin E1 and cyclin A2 as negative regulators of IDH1/2, 1DH1 and IDH2 accumulated in CCNE1^{-/-} and CCNA2^{-/-} MEFs, but not in CCNE2^{-/-}, CCND1^{-/-}, CCND2^{-/-} nor CCND3^{-/-} MEFs, accompanied with relatively higher oxidative phosphorylation rate in CCNE1^{-/-} MEFs (FIGS. 17D and 7C-7G). Notably, CDK6/cyclin D3 has been recently reported to inhibit glycolysis via directly phosphorylating PFKP and PKM2 (Wang et al., Nature 546, 426 (Jun. 15, 2017)). In contrast, an important role for CDK2/cyclin E1 and CDK2/cyclin A2 in suppressing TCA cycle was revealed, which was due, at least in part, to promoting the degradation of the TCA enzymes, IDH1/2. Without being bound by theory, these two mechanisms might represent complementary and synergistic molecular switches for tightly controlling the metabolism cycle in a cell cycle-dependent manner. As CDK2 can exert its kinase activity through binding either cyclin E or cyclin A (Koff et al., Science 257, 1689 (Sep. 18, 1992); Zhang et al., Cell 82, 915 (1995)), the remainder of the study explored the molecular mechanism underlying CDK2/cyclin E1-mediated degradation of IDH1/2.

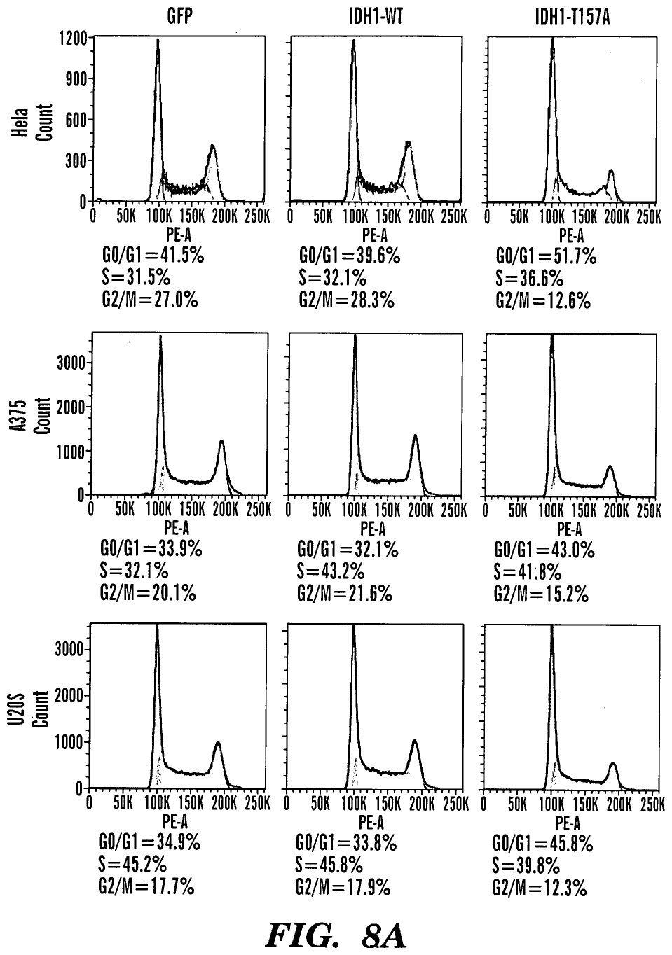

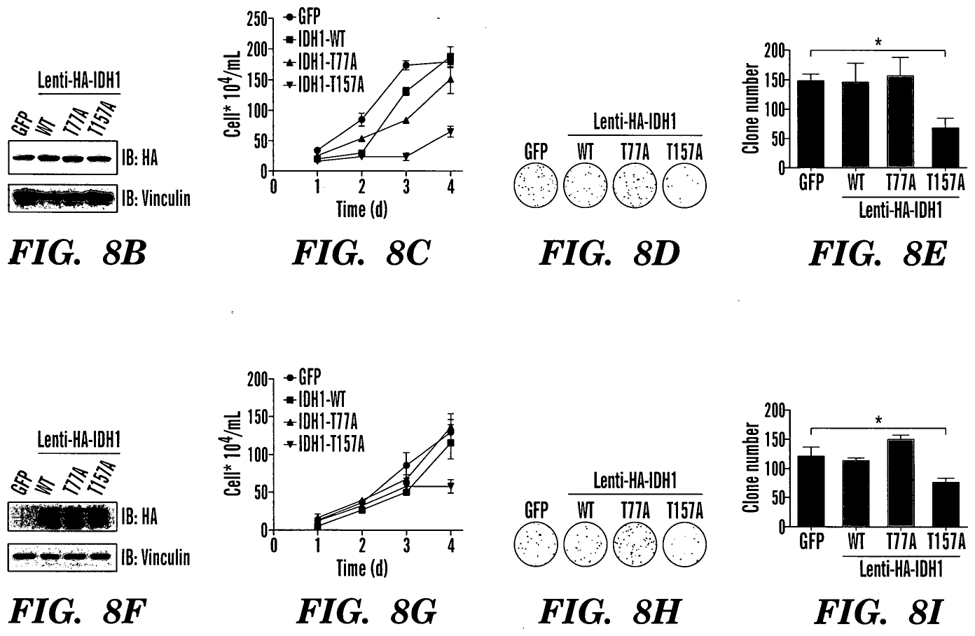

Importantly, the phosphorylation on T157 of exogenous IDH1 can be detected using mass spectroscopy (FIG. 7H). The T157 site is also conserved in mitochondrial IDH2 (T197). The Skp2/cyclin E/CDK2 signaling axis also negatively regulated IDH2 through this site, likely before newly synthesized IDH2 enters the mitochondria (FIG. 7B). In keeping with an important role for T157 in Skp2-mediated degradation of IDH1, mutating T157, but not the other two SP/TP motif residues T77 or S94, to alanine residues abolished cyclin E/CDK2-induced Skp2 interaction with recombinant IDH1 in vitro (FIG. 17E). Moreover, synthetic peptides with amino acid sequence derived from the putative phospho-degron region in IDH1 (T157) and IDH2 (T197) bound to recombinant Skp2, but not Fbw4 in vitro, only when T157 in IDH1 or T197 in IDH2 were phosphorylated (FIGS. 7I-7K). As a result, IDH1-T157A was not ubiquitinated by Skp2 in vivo (FIG. 17F) nor degraded by Skp2 in cells (FIGS. 17G and 7L-7M). Moreover, unlike IDH1-WT, the levels of ectopically expressed IDH1-T157A did not fluctuate during the cell cycle (FIG. 17H), which compromised the metabolic shift to glycolysis during the S phase (FIG. 17I-17J and 7N-7Q). The latter was associated with a modest increase in G1 cells (FIG. 8A), impaired proliferation, and decreased anchorage-independent growth in the soft agar, possibly due to impaired delivery of glycolytic intermediates needed for the robust assembly of biomass during S phase (FIGS. 17K-17O and 8B-8I).

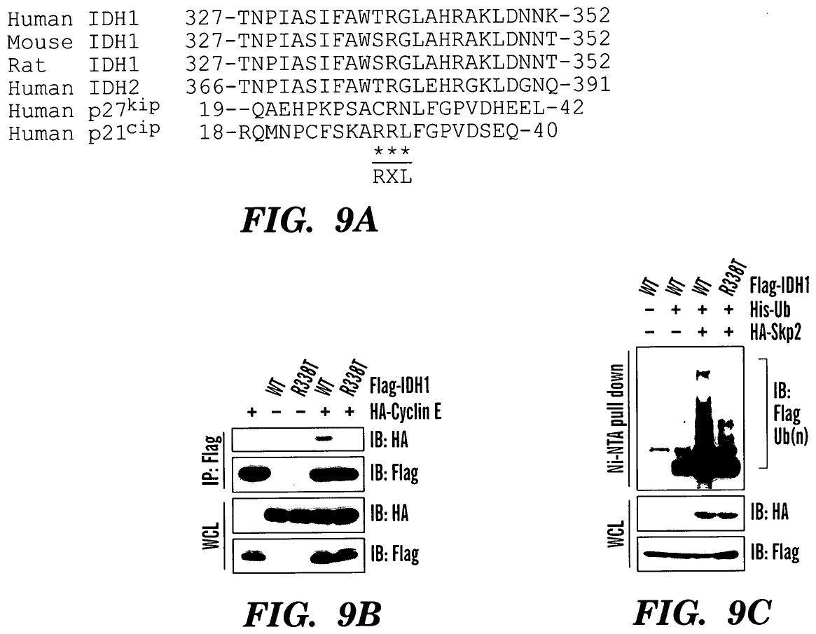

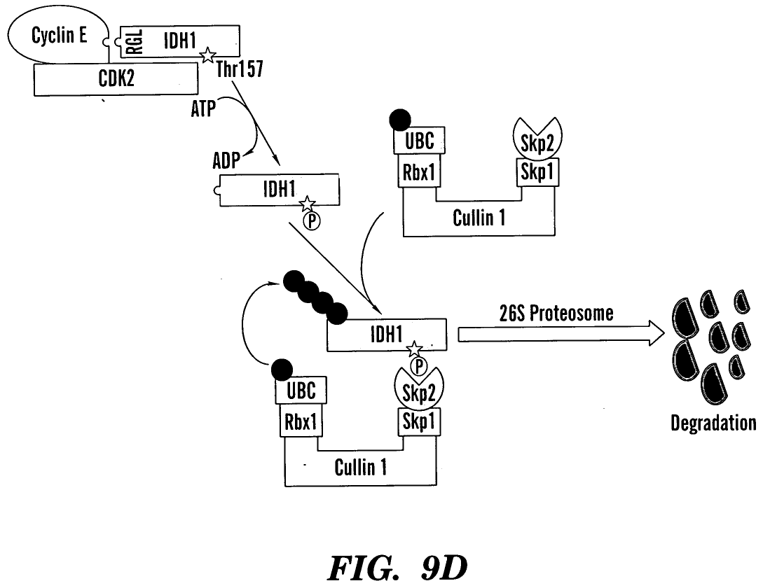

Previous studies revealed that numerous cyclin E substrates contain an RXL cyclin A/E-binding motif (Adams et al., Molecular and Cellular Biology 16, 6623 (1996)). Such an RXL motif was identified in both IDH1 and IDH2 (FIG. 9A), and found to be mutated in breast cancer (R338T) (Ciriello et al., Cell 163, 506 (Oct. 8, 2015)) and head and neck cancer (R338K) clinical samples (Morris et al., JAMA Oncol, (Jul. 21, 2016)). Notably, the cancer-derived R338T mutation abolished IDH1 interaction with cyclin E in cells (FIG. 9B), stabilizing the mutant form of IDH1 in part via escaping Skp2-mediated ubiquitination (FIG. 9C). Taken together, these findings indicate that IDH1 is phosphorylated by cyclin E/CDK2 presumably at least at the T157 residue, which is subsequently recognized by the SCF^{Skp2} E3 ubiquitin ligase for ubiquitination and subsequent degradation (FIG. 9D).

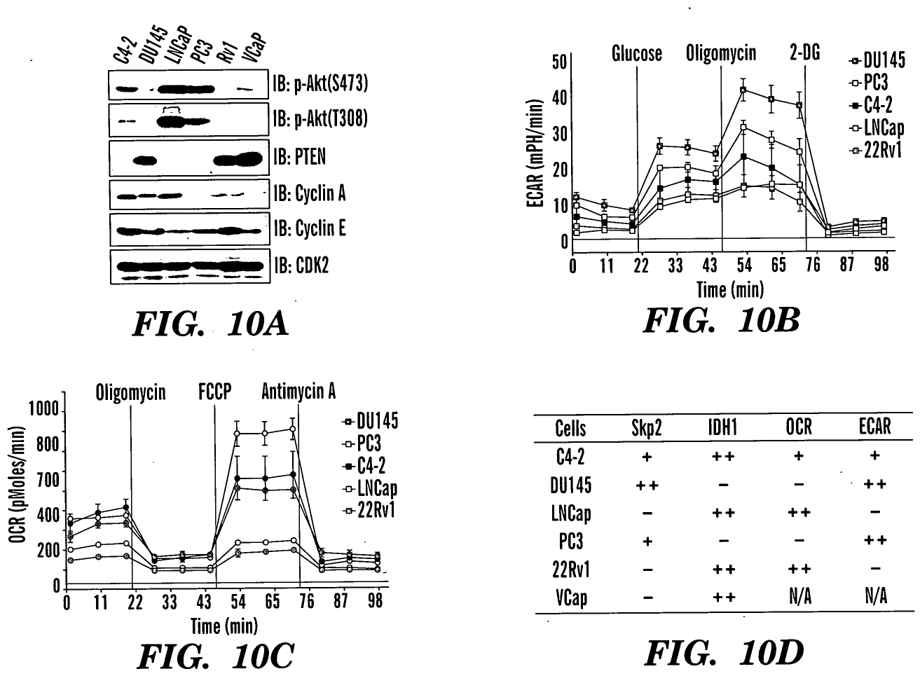

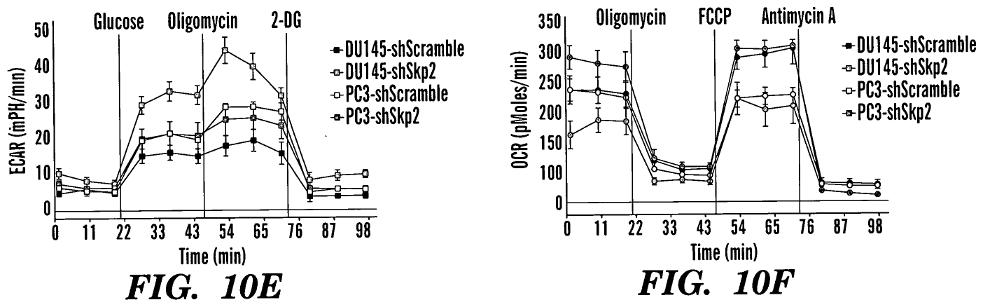

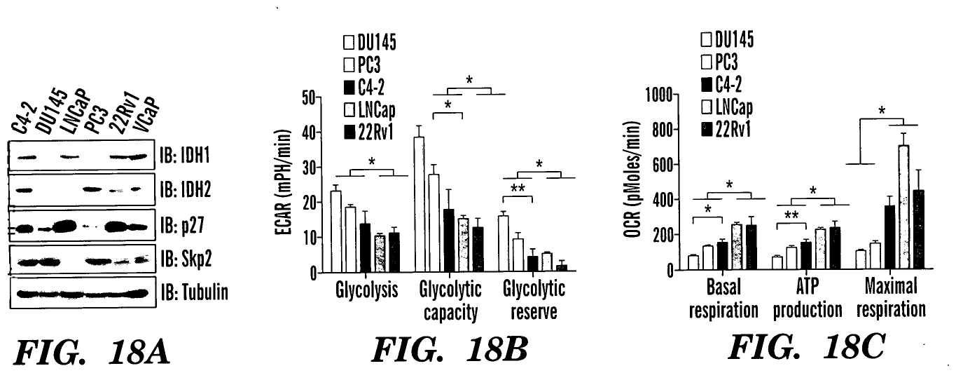

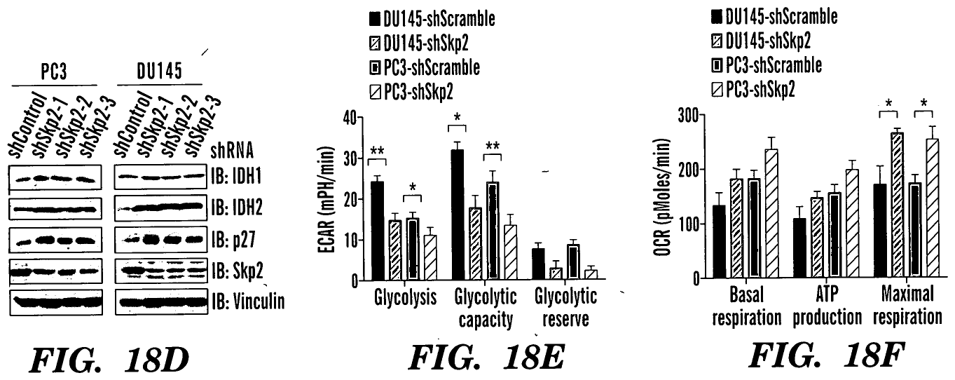

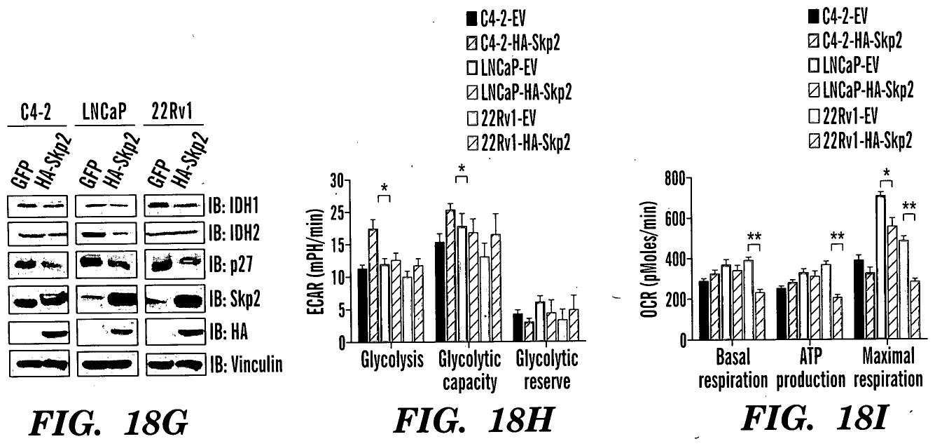

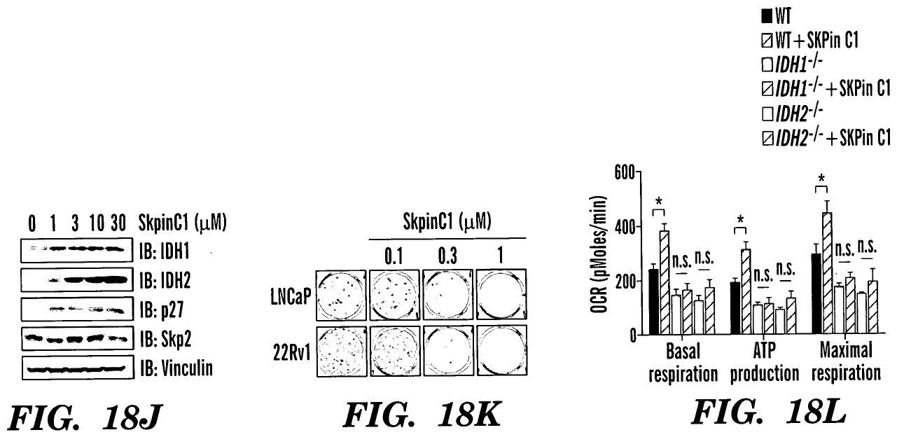

Skp2 plays an important role in prostate tumorigenesis (Lin et al., Nature 464, 374 (2010)). In keeping with an oncogenic role for Skp2, an inverse correlation between Skp2 and IDH1 expression was observed in a panel of prostate cancer (PrCa) cell lines (FIGS. 18A and 10A). In line with this finding, compared to the four PrCa cell lines featured Skp2^{low} and IDH1^{high} expression pattern (C4-2, LNCaP, VCaP and 22-Rv1), the two Skp2^{high} and IDH1^{low} PrCa cells (DU145 and PC3) displayed elevated rate of glycolysis (FIGS. 18B and 10B) and reduced rate of oxidative phosphorylation (FIGS. 18C and 10C-10D). Importantly, depletion of endogenous SKP2 in these two Skp2^{high} cells increased protein abundances of p27 and IDH1/2 (FIG. 18D), reduced glycolysis (FIGS. 18E and 10E) and increased oxidative phosphorylation (FIGS. 18F and 10F). On the other hand, enforced ectopic expression of Skp2 in Skp2^{low} cells, such as LNCaP, C4-2 and 22-Rv1, resulted in reduced p27 and IDH1/2 (FIG. 18G), increased glycolysis (FIG. 18H) and reduced oxidative phosphorylation (FIG. 18I). These results provide further support for an important role of Skp2 in negatively governing the protein stability of IDH1/2, and thereby coupling metabolism to cell cycle progression.

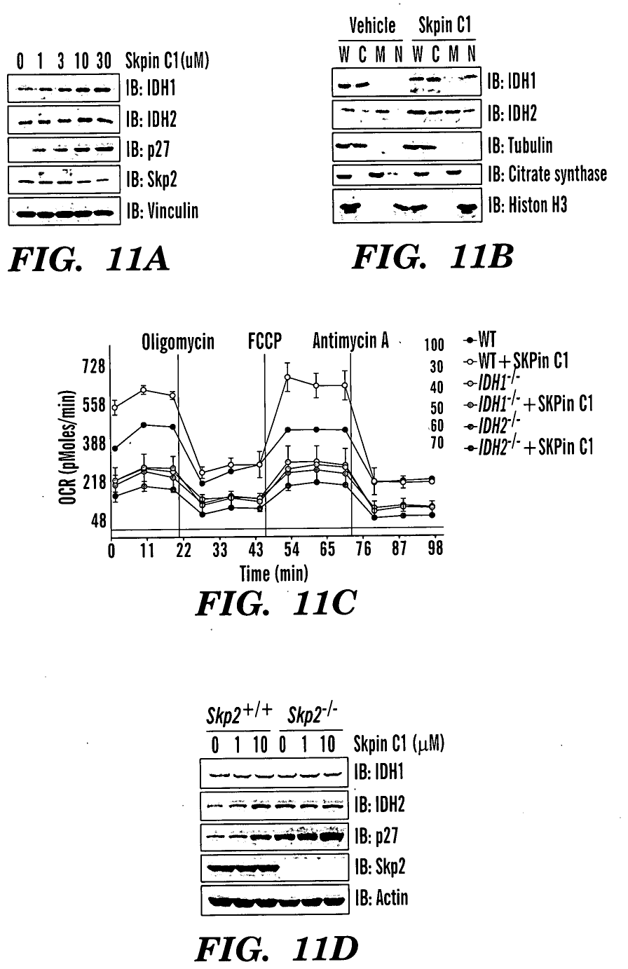

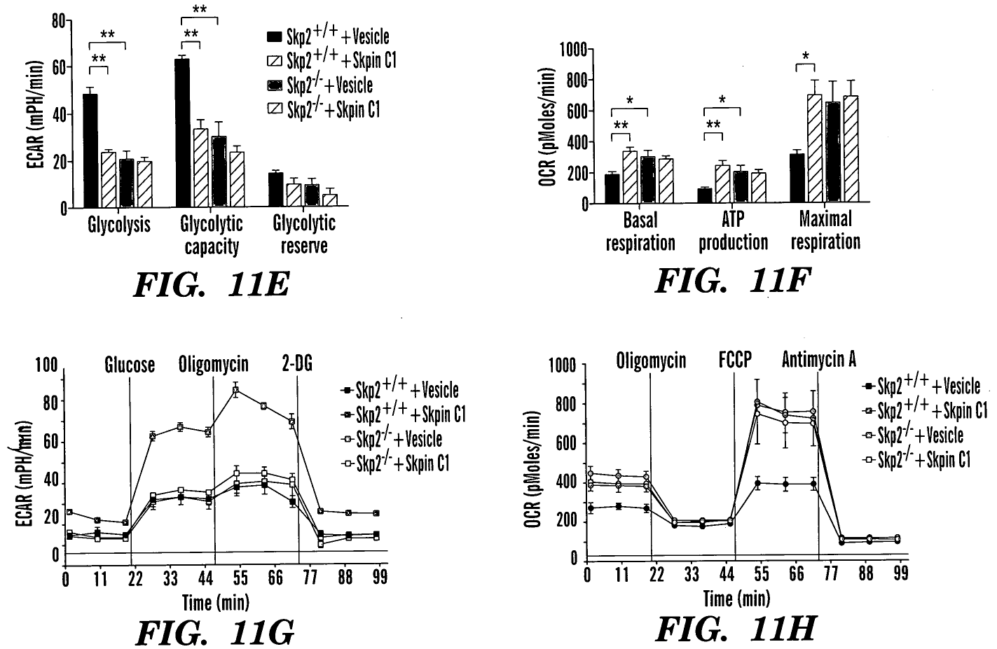

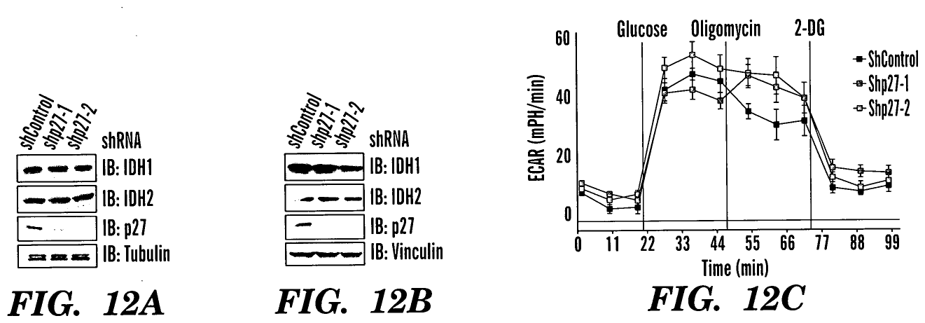

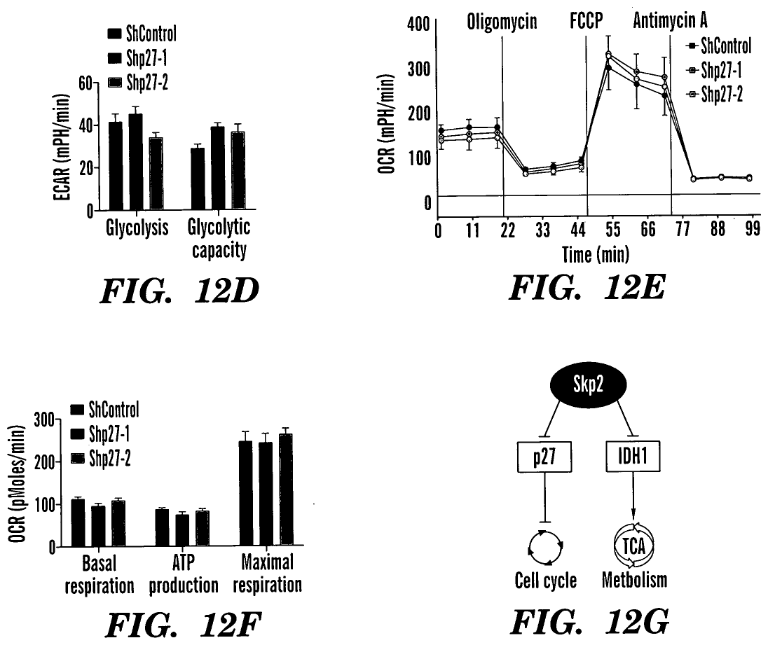

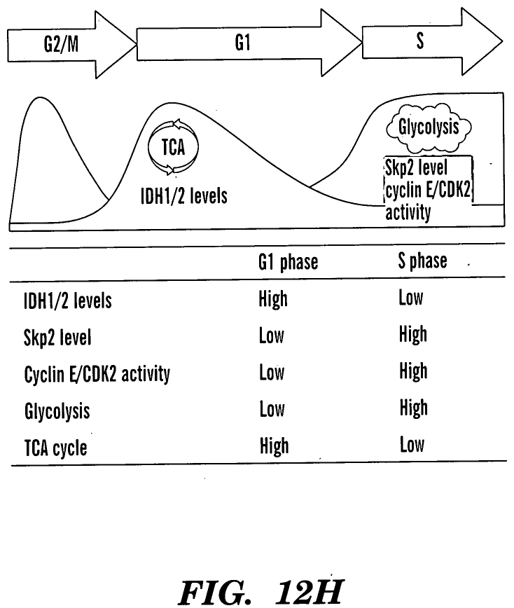

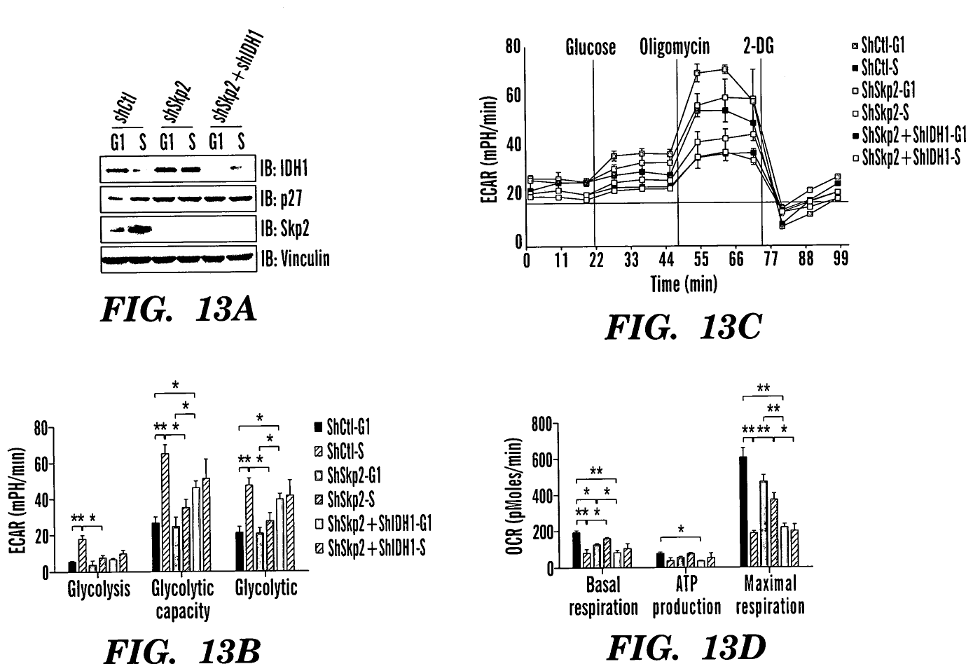

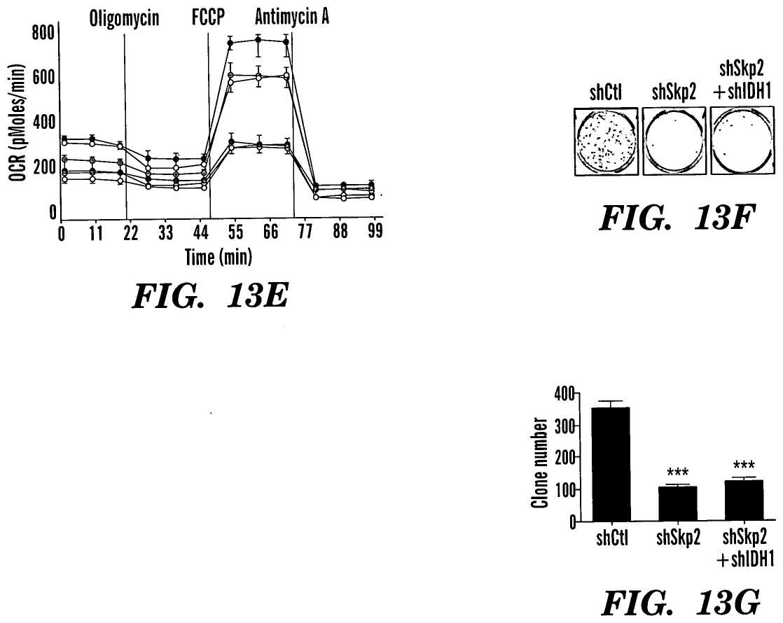

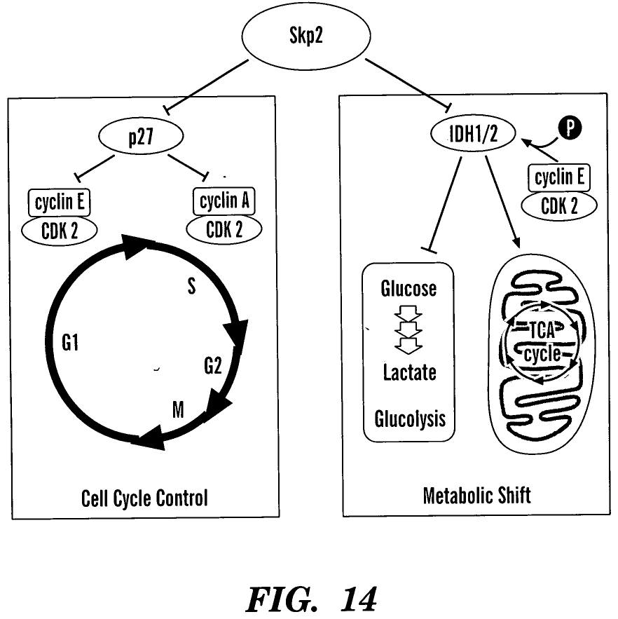

In keeping with this notion, treating 22-Rv1 and LNCaP cells with the Skp2 inhibitor, SKPin C1, which was developed as a selective inhibitor to block an interaction between Skp2 and p27 (Wu et al., Chemistry & biology 19, 1515 (2012)), significantly stabilized both IDH1 and IDH2 (FIGS. 18J and 11A). IDH1 was mainly localized in the cytoplasmic fraction regardless of SKPin C1 treatment (FIG. 11B). On the other hand, IDH2, which is normally mitochondrial, was detected in the cytoplasmic fraction after SKPin C1 treatment, suggesting that SCF^{Skp2}-mediated degradation of IDH2 possibly occurs before its translocation into the mitochondria (FIG. 11B). Moreover, SKPin C1 treatment phenocopied the effects of expressing the non-degradable T157A-IDH1 mutant with respect to cellular proliferation and metabolism (FIGS. 18K-18L and 11C). These effects were likely on-target because they were abolished in cells lacking SKP2 (FIGS. 11D-11H). p27 is one of the best characterized Skp2 ubiquitin substrates, which arrest cell cycle in G1 phase by inhibiting CDK kinase activities (Carrano et al., Nature cell biology 1, 193 (August 1999)). Interestingly, depletion of CDKN1B in multiple cell lines did not significantly affect the expression levels of IDH1/2 (FIGS. 12A-12B) or the metabolic phenotypes (FIGS. 12C-12F), thus excluding the possibility that the effects of Skp2 on IDH1/2 stability and the shift to glycolysis in S-phase were indirectly mediated by fluctuations in p27 protein abundance (FIGS. 12G-12H). In keeping with this notion, although depletion of SKP2 abolished the metabolic shift from TCA cycle to glycolysis in S phase (FIGS. 16 and 13A-13E), additional depletion of IDH1 in SKP2-depleted cells redirected cell metabolism in favor of glycolysis, even in G1 phase (FIG. 13A-13E). However, due to the accumulation of p27 and cell cycle blockage in SKP2-depleted cells, depletion of IDH1 did not reverse the effect of SKP2 depletion on colony formation (FIG. 13F-13G). Without being bound by theory, suppression of both p27-induced cell cycle arrest and IDH1-induced metabolism shift contributes to the oncogenic role of Skp2 (FIG. 14).

The present study defined a cell cycle-dependent metabolic cycle in mammalian cells, in part through SCF^{Skp2}-mediated IDH1 degradation (FIG. 16). Specifically, during the G1/S transition, accumulated cyclin E activates CDK2 (Koff et al., Science 257, 1689 (1992)), which in turn phosphorylates IDH1, leading to its recognition and ubiquitination by SCF^{Skp2} (FIG. 17). Moreover, in the prostate cancer settings, IDH1 protein abundance inversely correlates with Skp2, and the Skp2/IDH1 signaling axis drives the metabolic phenotypes (FIG. 18). The present study reveals a novel oncogenic role of Skp2 independent of its other biological substrate p27 in cell cycle regulation, by promoting the metabolic switch from utilization of TCA cycle to glycolysis. Thus, targeting Skp2 has the potential to provide a novel anti-cancer therapy in part by suppressing cancer metabolism.

The results described herein above, were obtained using the following methods and materials.

Plasmids and shRNAs

Skp2 cDNA was subcloned into CMV-GST, pcDNA3-HA and Lenti-puro-HA vectors via BamHI and XhoI sites. IDH1-WT cDNA were subcloned into pET28a-His, pGEX-GST, Flag-CMV and Lenti-hygro-HA vectors via BamHI and XhoI sites. Site directed mutagenesis to generate various IDH1 degron mutants were performed using the QuikChange XL Site-Directed Mutagenesis Kit (Stratagene) according to the manufacturer's instructions. HA-cyclin A, HA-cyclin E, HA-CDK2, HA-ERK1, HA-GSK3β and HA-Rbx1 were generated by cloning the corresponding cDNAs into pcDNA3-HA vector via BamHI and XhoI sites. CMV-GST-Fbl3a, CMV-GST-Fbl13, CMV-GST-Fbl18, CMV-GST-Fbo16, CMV-GST-β-TRCP1, CMV-GST-Fbw4, CMV-GST-Fbw6, CMV-GST-Fbw7 and CMV-GST-Skp2 were a kind gift of Dr. Wade Harper (Harvard Medical School). Myc-cullin 1, Myc-cullin 2, Myc-cullin 3, Myc-cullin 4A, Myc-cullin 4B and Myc-cullin 5 were a kind gift of Dr. James DeCaprio (Dana-Farber Cancer Institute). The lentiviral vectors containing Skp2 and p27 shRNAs were described before (Koff et al., Science 257, 1689 (1992)). The lentiviral vectors containing cullin 1, cullin 3 and Fbw4 shRNAs were purchased from Open biosystem.

### Antibodies

Anti-IDH1 (3997, 8137), anti-IDH2 (12652), anti-cullin 1(4995), anti-cullin 3 (2759), anti-cullin 4A (2699), anti-PTEN (9188), anti-Akt (pan) (2920), anti-pS473-Akt (4070), anti-pT308-Akt (8205), Anti-cyclin A2 (4656), anti-cyclin D1 (2978), anti-cyclin D2 (3741), anti-cyclin D3 (2936), anti-cyclin E1 (4129), anti-cyclin E2 (4132), anti-GST (2625), anti-p27 Kip (3698), anti-citrate synthase (14309), anti-aconitase (6922), anti-OGDH (13407), anti-succinyl-CoA synthetase (8071), anti-SDHA (11998), anti-fumarase (4567), anti-MDH2 (11908), anti-Myc-tag (2276, 2278) and anti-Histon H3 (4499) antibodies were purchased from Cell Signaling Technology. Anti-Skp2 (A-2, H435), anti-Plk1 (F-8), anti-Cdc20 (E-7), and polyclonal anti-HA (Y-11) antibodies were purchased from Santa Cruz. Anti-Tubulin (T-5168) and anti-Vinculin (V-4505) antibodies were purchased from Bethyl Labs. Polyclonal anti-Flag antibody (F-2425), monoclonal anti-Flag (F-3165) antibody, anti-Flag agarose beads (A-2220), anti-HA agarose beads (A-2095) as well as peroxidase-conjugated anti-mouse secondary antibody (A-4416) and peroxidase-conjugated anti-rabbit secondary antibody (A-4914) were purchased from Sigma. Monoclonal anti-HA antibody (MMS-101P) was purchased from Covance. Anti-GFP antibody (632380) and polyclonal anti-Cdh1 antibody (34-2000) were purchased from Invitrogen. Anti-Fbw4 antibody (60116) was purchased from Abcam.

### Cell Culture and Transfection

Human embryonic kidney 293 (HEK293) cells, HEK293FT, HeLa, DLD1, HCT116, U205, T98G, A375, VCaP, HAP1 cells and mouse embryonic fibroblasts (MEFs) were maintained in Dulbecco's Modified Eagle's Medium (DMEM) containing 10% fetal bovine serum (FBS), 100 Units of penicillin and 100 mg/ml streptomycin. PC3, DU145, 22Rv1, LNCaP and C4-2 cells were cultured in RPMI1640 containing 10% fetal bovine serum (FBS), 100 Units of penicillin and 100 mg/ml streptomycin. RWPE cells were maintained in keratinocyte serum free medium (K-SFM, Invitrogen, 44019). Skp2^{+/+} and Skp2^{-/-} MEFs were described previously (Inuzuka et al., Cell 150, 179-193 (2012)). Cyclin A2^{f/f}, cyclin E1^{-/-}E2^{-/-}, cyclin E1^{-/-}, cyclin E2^{-/-}, cyclin D1^{-/-}, cyclin D2^{-/-} and cyclin D3^{-/-} MEFs were a kind gift of Dr. Piotr Sicinski (Dana Farber Cancer Institute). HAP1-IDH1^{-/-} (HZGHC003323c006) and HAP1-IDH2^{-/-} (HZGHC000919c010) cells were purchased from Horizon Discovery. HAP1 is a near-haploid human cell line, which was derived from KBM-7, a chronic myelogenous leukemia (CML) cell line (Carette et al., Science 326, 1231-1235). HeLa-IDH1^{-/-} cells were generated using CRISPR/Cas 9 with a guide sequence of 5′-TACGAAATATTCTGGGTGGC-3′ (Sanjana et al., Nature methods 11, 783-784 (2014)). Cell culture transfection, lentiviral virus packaging and subsequent infection of various cell lines were performed according to the protocol described previously (Boehm et al., Molecular and cellular biology 25, 6464-6474 (2005)). To determine the proliferation ability of HAP1 after depletion of IDH1 or IDH2, cells were cultured in H-DMEM, then transferred into DMEM media without glucose (Thermo Fisher, 11966025) after adding either 25 mM of D-glucose (Sigma, G8270) or D-galactose (Sigma, G0750).

HeLa and HCT116 cells were used for synchronization. HeLa cells, which have low endogenous Skp2 activity, were used for ectopic expression-based degradation assays. HEK293 cell line was used for ubiquitination assays and co-IP assays to define the interaction between two ectopically expressed proteins, which is the most frequently used cell line for this type of experiment. Human prostate cancer cells, DU145, PC3, LNCaP, VCaP, 22Rv1 and C4-2 were used for compared endogenous Skp2 and IDH1 levels, as well as Skp2 knockdown and Skp2 overexpression. HAP1, LNCaP, and 22Rv1 cells were also used for treating with Skp2 inhibitor, SKPin C1 (MCE, HY-16661).

### Cell Synchronization

Cell synchronization with nocodazole arrest was described previously (Wan et al., Developmental cell 29, 377-391 (2014); Wei et al., Nature 428, 194-198 (2004)). Briefly, HeLa cells or HCT116 cells were incubated with 10 μg/mL for 20 hours, followed by knocking the dish on a hard surface to dislodge mitotic cells, and washing with PBS for 3 times. The cells were released at the indicated times before harvest.

### Seahorse XF24 Extracellular Bioenergetics Analysis

Oxygen consumption rate (OCR) and extracellular acidification rate (ECAR) were measured using Seahorse XF24 analyzer (Boston, Mass., USA). OCR assays used Seahorse XF basal media containing 25 mM glucose, 1 mM sodium pyruvate, and 2 mM glutamine, while ECAR assays used Seahorse XF basal media containing no glucose, no pyruvate, and 2 mM glutamine. For OCR assays, the final concentrations of oligomycin, FCCP, and antimycin A were 1, 0.3, and 1 μM, unless indicated otherwise. For ECAR assays, the final concentrations of glucose, oligomycin, and 2-DG were 10 mM, 1 μM, and 50 mM, unless indicated otherwise.

### Immunoblots (IB) and Immunoprecipitation (IP)

Cells were lysed in EBC buffer (50 mM Tris pH 7.5, 120 mM NaCl, 0.5% NP-40) supplemented with protease inhibitors (cOmplete Mini, Roche) and phosphatase inhibitors (phosphatase inhibitor cocktail set I and II, Calbiochem). The protein concentrations of the lysates were measured using the Bio-Rad protein assay reagent on a Beckman Coulter DU-800 spectrophotometer. The lysates were then resolved by SDS-PAGE and immunoblotted with indicated antibodies. For immunoprecipitation, 1 mg lysates were incubated with the appropriate sepharose beads for 4 hours at 4° C. Immuno-complexes were washed four times with NETN buffer (20 mM Tris, pH 8.0, 100 mM NaCl, 1 mM EDTA and 0.5% NP-40) before being resolved by SDS-PAGE and immunoblotted with indicated antibodies.

### In Vitro Kinase Assay

IDH1 in vitro kinase assays were performed as previous reported (Liu et al., Nature 508, 541-545 (2014)). Briefly, His-IDH1 was expressed in BL21 E. coli and purified using Ni-NTA (Ni-nitrilotriacetic acid) agarose (Thermo Fisher Scientific) according to the manufacturer's instructions. One microgram of His-IDH1 WT or mutant protein was incubated in the absence or presence of Cyclin E/Cdk2 kinase in kinase assay buffer (10 mM HEPES, pH 8.0, 10 mM MgCl₂, 1 mM dithiothreitol, 0.1 mM ATP). The reaction was initiated by the addition of 10× kinase assay buffer in a volume of 30 μl for 45 min at 30° C. followed by the addition of SDS-PAGE sample buffer to stop the reaction before resolved by SDS-PAGE.

### In Vitro Pull Down Assay

His-Skp2 and GST-IDH1 were expressed in BL21 E. coli and purified using Ni-NTA agarose or Glutathione Sepharose 4B (GE Healthcare Life Sciences) according to the manufacturer's instructions. The GST-IDH1 proteins (2 μg) were eluted using elution buffer (50 mM Tris-HCl, pH 8.0, 10 mM reduced glutathione) and incubated with or without cyclin E/Cdk2 in kinase assay buffer for 1 hour. Then, the reaction solution was added with His-Skp2 beads (1 μg) and incubated at 4° C. for 3 hours followed by the addition of SDS-PAGE sample buffer to stop the reaction before resolved by SDS-PAGE.

### In Vivo Ubiquitination Assays

Denatured in vivo ubiquitination assays were performed as previous described (Wei et al., Nature 428, 194-198 (2004)). Briefly, HEK293 cells were transfected with Flag-IDH1, His-ubiquitin and HA-Skp2. 48 hours after transfection, 30 μM MG132 was added to block proteasome degradation for 6 hours and cells were harvested in denatured buffer (6 M guanidine-HCl, pH 8.0, 0.1 M Na₂HPO4/NaH₂PO4, 10 mM imidazole). After sonication, the ubiquitinated proteins were purified by incubation with Ni-NTA matrices for 3 hours at room temperature. The pull-down products were washed sequentially twice in buffer A, twice in buffer ANTI mixture (buffer A: buffer TI=1:3) and once in buffer TI (25 mM Tris-HCl, pH 6.8, 20 mM imidazole). The poly-ubiquitinated proteins were separated by SDS-PAGE for immunoblot analysis.

### FACS Analysis

Cells synchronized with nocodazole-arrest and release were collected at the indicated time points and stained with propidium iodide (Roche) according to the manufacturer's instructions. Stained cells were sorted with a Dako-Cytomation MoFlo sorter (Dako) at the Dana-Farber Cancer Institute FACS core facility.

### Peptide-Binding Assays

The IDH1 peptides with/without phosphorylation modification were synthesized by LifeTein, LLC (Somerset, N.J.). Each peptide contained an N-terminal biotin and free C-terminus. The peptides were diluted into 1 mg/ml for further biochemical assays. The sequences are listed below:

| | |
|---|---|
| IDH1 | Biotin-TDFVVPGPGKVEITYTPSDGTQKVTYLVHNF |
| pIDH1 | Biotin-TDFVVPGPGKVEITYT(p)PSDGTQKVTYLVHNF |
| IDH2 | Biotin-TDFVADRAGTFKMVFTPKDGSGVKEWEVYNF |
| pIDH2 | Biotin-TDFVADRAGTFKMVFT(p)PKDGSGVKEWEVYNF |

Peptides (2 μg) were incubated with 10 μg of recombinant SKP2 proteins for 4 hours at 4° C., 10 μl Streptavidin agarose (GE Healthcare Life Sciences) was added in the sample for another 1 hour. The agarose was washed four times with NETN buffer. Bound proteins were added in 2×SDS loading buffer and resolved by SDS-PAGE for immunoblot analysis.

### Mass Spectrometry Analysis

The procedures of mass spectrometry analysis were performed as described previously with minor modifications (Liu et al., Nature 508, 541-545 (2014)). Briefly, anti-Flag-IDH1 immunoprecipitations were performed with the whole cell lysates derived from three 10 cm dishes of HEK293 cells co-transfected with Flag-IDH1, HA-cyclin E and HA-CDK2. The immunoprecipitations proteins were resolved by SDS-PAGE, and stained by Gelgold staining buffer. The band containing IDH1 was reduced with 10 mM DTT for 30 min, alkylated with 55 mM iodoacetamide for 45 min, and in-gel-digested with trypsin enzymes. The resulting peptides were extracted from the gel and analyzed by microcapillary reversed-phase liquid chromatography-tandem mass spectrometry (LC-MS/MS) using a high resolution Orbitrap Elite (Thermo Fisher Scientific) in positive ion DDA mode via CID, as previously described. MS/MS data were searched against the human protein database using Mascot (Matrix Science) and data analysis was performed using the Scaffold 4 software (Proteome Software).

### Clonogenic Survival and Soft Agar Assay

Cells were cultured in 10% FBS containing DMEM or RPMI-1640 media before plating into 6-well plate at 10,000 cells (3,000 cells for HeLa) per well. Ten days later, cells were fixed with 10% acetic acid/10% methanol for 10 min, stained with 0.4% crystal violet/20% ethanol, followed by counting the colony numbers. For soft agar assays, cells were seeded in 0.4% low-melting-point agarose in DMEM or RPMI-1640 with 10% FBS at 100,000 per well (30,000 cells for HeLa), layered onto 0.8% agarose in DMEM or RPMI-1640 with 10% FBS. The plates were kept in the cell culture incubator for 3-4 weeks after which the cells were stained with iodonitrotetrazolium chloride and colonies were counted.

### Unlabeled and Labeled Metabolites Extraction

U-¹³C6-glucose-labeled DMEM medium was prepared with non-glucose, non-glutamine and non-pyruvate DMEM media by adding 10 mM of U-¹³C6 D-glucose (Cambridge Isotope Laboratories), 1 mM sodium pyruvate and 2 mM glutamine. U-¹³C5-glutamine-labeled DMEM medium was prepared with non-glucose, non-glutamine and non-pyruvate DMEM media by adding 2 mM of U-¹³C5 glutamine (Cambridge Isotope Laboratories), 1 mM sodium pyruvate and 25 mM glucose.

Unlabeled and ¹³C-labeled flux assays were performed according as previously reported (Wan et al., Developmental cell 29, 377-391 (2014)). Briefly, media was refreshed one hour before harvesting cells to remove accumulated metabolic wastes. For metabolites labeling, before harvesting sample, media were changed to U-¹³C6-glucose-labeled media for labeling for 30, 60 and 120 seconds or U-¹³C5-glutamine-labeled media for labeling for 1, 2 and 3 hours. Then the media was aspirated completely and 4 ml of dry ice-cold 80% MeOH was added, followed by placing the plates at −80° C. for 30 minutes. Then the metabolites were extracted as previously described and normalized by protein amount. All metabolites were analyzed as previously described (Yuan et al., Nature protocols 7, 872-881 (2012)).

### Glucose and Glutamine Uptake

The uptakes of glucose and glutamine for HeLa cells in either G1 phase or S phase were measured using Glucose Uptake Cell-Based Assay Kit (600470, Cayman Chemical) and Glutamate Assay kit (ab83389, Abcam) according to the manufacturer's protocol. For glucose uptake, cells were stained with 2-NBDG followed by flow cytometry analysis (excitation/emission=485/538 nm). For glutamine uptake, cells were harvested and analyzed at OD₄₅₀.

### Fraction of Cytoplasm, Mitochondria and Nuclei

Cells were harvested and subjected to fractionation of cytoplasm (C), mitochondria (M), and nuclei (N) using Cell Fractionation kit (ab109719, Abcam). All fractions and the whole cell lysate (WCL) were subjected to immunoblot analysis for the indicated proteins, with tubulin, citrate synthase, and Histone H3 as markers of cytoplasm, mitochondria, and nucleus, respectively.

### Statistical Analysis

The quantitative data from multiple repeat experiments were analyzed by a two-tailed unpaired Student's t test or one-way ANOVA, and presented as mean±s.e.m. When P<0.05, the data were considered as statistically significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of reducing neoplastic cell proliferation or survival, the method comprising contacting the cell with a Skp2 inhibitor and a Pyruvate kinase M2 (PKM2) inhibitor, thereby reducing neoplastic cell proliferation or survival.

2. A method of reducing tumor growth, the method comprising contacting the tumor with a Skp2 inhibitor and a Pyruvate kinase M2 (PKM2) inhibitor, thereby reducing tumor growth.

3. A method of treating cancer in a subject, the method comprising administering to the subject a Skp2 inhibitor and a Pyruvate kinase M2 (PKM2) inhibitor, thereby treating cancer in the subject.

4. The method of claim 1, wherein the neoplastic cell or tumor displays increased glycolytic metabolism.

5. The method of claim 1, wherein the neoplastic cell or tumor displays reduced Tricarboxylic Acid (TCA) metabolism.

6. The method of claim 1, wherein the neoplastic cell or tumor displays increased lactate production.

7. The method of claim 1, wherein the neoplastic cell or tumor is characterized as Skp2^{high} and IDH1^{low}.

8. The method of claim 1, wherein the neoplastic cell or tumor displays reduced oxidative phosphorylation.

9. The method of claim 1, wherein the neoplastic cell is a breast cancer, glioblastoma, or prostate cancer cell.

10. The method of claim 1, wherein the tumor is breast cancer, glioblastoma, or prostate cancer.

11. A method of treating a selected subject having cancer, the method comprising administering a Skp2 inhibitor and an inhibitor of a glycolysis pathway enzyme to a selected subject, wherein the subject is selected by detecting an increased level of Skp2 and a decreased level of IDH1 and/or IDH2 in a biological sample of the subject, thereby treating the subject.

12. The method of claim 11, wherein the subject has breast cancer, glioblastoma, or prostate cancer.

13. The method of claim 11, wherein the subject's cancer displays increased glycolytic metabolism.

14. The method of claim 11, wherein the subject's cancer displays reduced Tricarboxylic Acid (TCA) metabolism.

15. The method of claim 11, wherein the neoplastic cell or tumor displays increased lactate production.

16. The method of claim 11, wherein the neoplastic cell or tumor displays reduced oxidative phosphorylation.

17. The method of claim 11, wherein Skp2, p27, p21, Cyclin A, Cyclin E, IDH1, and/or IDH2 expression is detected by immunoassay.

18. A therapeutic combination for cancer therapy comprising a Skp2 inhibitor and a PKM2 inhibitor.

19. The combination of claim 18, wherein the Skp2 inhibitor and PKM2 inhibitor are formulated together or separately.

20. (canceled)

21. The method of claim 1, wherein the PKM2 inhibitor is one or more of 2 inhibitor compound 3k, DASA-58, and an inhibitory nucleic acid that targets PKM2 mRNA.


SEQUENCE LISTING (32 sequences)

SEQ ID NO: 1 - protein, 424 aa, Homo sapiens
MHRKHLQEIPDLSSNVATSFTWGWDSSKTSELLSGMGVSALEKEEPDSENIPQELLSNLGHPESPPRKRLKSKGSDKDFVIVRRPKLNRENFPGVSWDSLPDELLLGIFSCLCLPELLKVSGVCKRWYRLASDESLWQTLDLTGKNLHPDVTGRLLSQGVIAFRCPRSFMDQPLAEHFSPFRVQHMDLSNSVIEVSTLHGILSQCSKLQNLSLEGLRLSDPIVNTLAKNSNLVRLNLSGCSGFSEFALQTLLSSCSRLDELNLSWCFDFTEKHVQVAVAHVSETITQLNLSGYRKNLQKSDLSTLVRRCPNLVHLDLSDSVMLKNDCFQEFFQLNYLQHLSLSRCYDIIPETLLELGEIPTLKTLQVFGIVPDGTLQLLKEALPHLQINCSHFTTIARPTIGNKKNQEIWGIKCRLTLQKPSCL

SEQ ID NO: 2 - DNA, 3520 bp, Homo sapiens
aattcccagcaggccttgggcctcagtgcggccgcgaagcagagcgggctgtagagccttgcgcgcgcagtggggatggaacgttgctaggcttagcgggtctggctgctgggggcccgagcagcacgctcggagccgccgcgcgccaaagcgggaatctgggaggcgagcagctctgcagttaatgcacgtattttaaactcccgggcctgcggacgctatgcacaggaagcacctccaggagattccagacctgagtagcaacgttgccaccagcttcacgtggggatgggattccagcaagacttctgaactgctgtcaggcatgggggtctccgccctggagaaagaggagcccgacagtgagaacatcccccaggaactgctctcaaacctgggccacccggagagccccccacggaaacggctgaagagcaaagggagtgacaaagactttgtgattgtccgcaggcctaagctaaatcgagagaactttccaggtgtttcatgggactcccttccggatgagctgctcttgggaatcttttcctgtctgtgcctccctgagctgctaaaggtctctggtgtttgtaagaggtggtatcgcctagcgtctgatgagtctctatggcagaccttagacctcacaggtaaaaatctgcacccggatgtgactggtcggttgctgtctcaaggggtgattgccttccgctgcccacgatcatttatggaccaaccattggctgaacatttcagcccttttcgtgtacagcacatggacctatcgaactcagttatagaagtgtccaccctccacggcatactgtctcagtgttccaagttgcagaatctaagcctggaaggcctgcggctttcggatcccattgtcaatactctcgcaaaaaactcaaatttagtgcgacttaacctttctgggtgttctggattctctgaatttgccctgcagactttgctaagcagctgttccagactggatgagctgaacctctcctggtgttttgatttcactgaaaagcatgtacaggtggctgttgcgcatgtgtcagagaccatcacccagctgaatcttagcggctacagaaagaatctccagaaatcagatctctctactttagttagaagatgccccaatcttgtccatctagacttaagtgatagtgtcatgctaaagaatgactgctttcaggaatttttccagctcaactacctccaacacctatcactcagtcggtgctatgatataatacctgaaactttacttgaacttggagaaattcccacactaaaaacactacaagtttttggaatcgtgccagatggtacccttcaactgttaaaggaagcccttcctcatctacagattaattgctcccatttcaccaccattgccaggccaactattggcaacaaaaagaaccaggagatatggggcatcaaatgccgactgacactgcaaaagcccagttgtctatgaagtatttattgcaggatggtgtctcttctttagaacagggaaaataggcaggaagcccaattgctggagtacttagctagttttattcttggttttccctttgccttcattctgcaagtatactagggagccatttgagagggaaaactatgaaatcttgctttttgaaatgattctaaaagcttctatcactgctttgctcttaagagccaaagttgtaggccttttgaaattttaggagagtgagcctataatttcaagataccttaaagagcaaaatttgagccacctcttccaagtgcccttcttactaagtctattcagaatcaagcttaaaaattaccaccagcaaacaatcttcatagcccatataacttttatctatttaattttatagtattgctttataagacagcttagaagaacaataagctatttgtattatgagctgaacaaaaagagaatcataggatagtagcgtctgaggccatcttttctaggaataggaaagagaaaaatgtatttgaattttgcctttagatttgaaattaggttaatagaaataagtaaccccatgtaattcaccttaaaacttaacaaaagaccaaacattacaaaacccagagatatagaatcaatataggatttgaaggcccagcagacagttttctatgacaggttaatctgaagtatcctgtaatgttcattaagttactgtgtttccagaatctaaattagatgagaaatataattgtggttttctaacttgataatcaaattatgttaacatgggtcctttagcttttaaaatgacttgctttgttttagaaaggtggtattaatccactctctattcttgaaaatttggatgggagaattctgaagttgcctgctgttttcctttagcgctgaggttcttaaggttacttttatattactctggaatcaagtattttaaattgtattttttttttaaatgatctctcagcaataattgtttgaaactatccatatataaggttatcagacctacagttccctaagaggaactgcatgttctcttcaatcagaaatatacagtagaagcaggtatatcttccatgcagtttcagtagtaagcactacttatacctacataagagttaaaatccagatgtgggaccttttgataccatcagtgatatatatttttttaaactggtacagagaagtgaaaagattaaattctacttctattttttttttttttttttgagacggagtctcgctctgtcaccaaggccggagtgcagtggtgcgatctcggctcactgcaagctccgcctcccaggttcacgtcattctcctgcctcagcctcccgactagctgggactacaggcgcccaccaccacgcccggctaatttttttgtatttttagtagagacggggtttcaccatgttagccaggatggtctcaatctcctgacctcatgatccgcccgtcttggcctcccaaagtgctgggattacaggcatgagcaactgcgcccagccaaattctacttcttaaaaatcacaaaaactagtttaaattgatgacttgttcgtatgttcaaaatgtaacaacaaaaaaagctaacaccagtcatttatattaactttttttttttaaatcaaaaattgttaatgttagaaacatactatgaagtgcctttatctgcttagacctaaggaagattttaaagttgggttgcacaggaaatgatgatgcttcaatttcttaatagttaaaaagtgctaaatactacttgaaattattgtttacagattagtgacaagagctggggttaggatccggttggactctgacatcggatgccctcaaacatacagaacttccaaactcaagtccagccataagctattttgccaacatgtcagagtaatctgtatttttgtatgtgatttctacttttatagacttgttttaaaacaataaaacacatttttataaaaatgagtgcttaaaaaaaaaaaaaaaaaa

SEQ ID NO: 3 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 4 - DNA, 2402 bp, Homo sapiens
gggctgaggaggcggggcctgggaggggacaaagccgggaagaggaaaagctcggacctaccctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccattaaaaaaaaaa

SEQ ID NO: 5 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

SEQ ID NO: 6 - DNA, 1818 bp, Homo sapiens
tccccggcaaggcccaatggggcggcaggcccggcagccccgccccggtggtgcccgcgcggccagcgcccgccaggcccagcgttagcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaaaaaaaaa

SEQ ID NO: 7 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 8 - DNA, 2754 bp, Homo sapiens
aacccataaatctgggccctgcccaggtaggccgggacagctggggtggcctgggccgagagccaagaaaagacaccccatctggcagcccaacttggcggcaacaggtggcccggcgcccgggggtctgggaggaaagtcgctccgggggcgggccccgttgccccgccgcgtccccattggtcatcaggtttcttaaaatgtgactctgaatctgtgtccttccgccgcagaatttagtcccaccgaaagggcaacctgcccgcgcgttccgccaccgccgccgcgcttcctcctgaaggtgactgcgcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcgccggaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcaccaaaaaaaaaaaaaaa

SEQ ID NO: 9 - protein, 22 aa, Homo sapiens
TITPDEKRVEEFKLKQMWKSPN

SEQ ID NO: 10 - protein, 22 aa, Mus sp.
TITPDEKRVEEFKLKQMWKSPN

SEQ ID NO: 11 - protein, 22 aa, Rattus sp.
TITPDEKRVEEFKLKQMWKSPN

SEQ ID NO: 12 - protein, 22 aa, Homo sapiens
TITPDEKRVEEFKLKKMWKSPN

SEQ ID NO: 13 - protein, 22 aa, Mus sp.
TITPDEKRVEEFKLKKMWKSPN

SEQ ID NO: 14 - protein, 22 aa, Rattus sp.
TITPDEKRVEEFKLKKMWKSPN

SEQ ID NO: 15 - protein, 12 aa, Homo sapiens
GKVEITYTPSDG

SEQ ID NO: 16 - protein, 12 aa, Mus sp.
GKVEITYTPKDG

SEQ ID NO: 17 - protein, 12 aa, Rattus sp.
GKVEITYTPKDG

SEQ ID NO: 18 - protein, 12 aa, Homo sapiens
GTFKMVFTPKDG

SEQ ID NO: 19 - protein, 12 aa, Mus sp.
GTFKMVFTPKDG

SEQ ID NO: 20 - protein, 12 aa, Rattus sp.
GTFKMVFTPKDG

SEQ ID NO: 21 - protein, 31 aa, Artificial Sequence
  Description of Artificial Sequence Synthetic polypeptide
  N-term Biotin
TDFVVPGPGKVEITYTPSDGTQKVTYLVHNF

SEQ ID NO: 22 - protein, 31 aa, Artificial Sequence; identical to SEQ ID NO: 21
  Description of Artificial Sequence Synthetic polypeptide
  N-term Biotin
  MOD_RES (16)..(16): PhosphoThr

SEQ ID NO: 23 - protein, 31 aa, Artificial Sequence
  Description of Artificial Sequence Synthetic polypeptide
  N-term Biotin
TDFVADRAGTFKMVFTPKDGSGVKEWEVYNF

SEQ ID NO: 24 - protein, 31 aa, Artificial Sequence; identical to SEQ ID NO: 23
  Description of Artificial Sequence Synthetic polypeptide
  N-term Biotin
  MOD_RES (16)..(16): PhosphoThr

SEQ ID NO: 25 - protein, 13 aa, Unknown
  Description of Unknown IDH1 sequence
  MOD_RES (6)..(6): PhosphoThr
VEITYTPSDGTQK

SEQ ID NO: 26 - protein, 24 aa, Homo sapiens
TNPIASIFAWTRGLAHRAKLDNNK

SEQ ID NO: 27 - protein, 24 aa, Mus sp.
TNPIASIFAWSRGLAHRAKLDNNT

SEQ ID NO: 28 - protein, 24 aa, Rattus sp.
TNPIASIFAWSRGLAHRAKLDNNT

SEQ ID NO: 29 - protein, 24 aa, Homo sapiens
TNPIASIFAWTRGLEHRGKLDGNQ

SEQ ID NO: 30 - protein, 22 aa, Homo sapiens
QAEHPKPSACRNLFGPVDHEEL

SEQ ID NO: 31 - protein, 21 aa, Homo sapiens
RQMNPCFSKARRLFGPVDSEQ

SEQ ID NO: 32 - DNA, 20 bp, Artificial Sequence
  Description of Artificial Sequence Synthetic oligonucleotide
tacgaaatattctgggtggc